(19) United States
(12) Patent Application Publication
(10) Pub. No.: US 2015/0051536 A1
(43) Pub. Date: Feb. 19, 2015

(54) EXTENSIONLESS REVERSIBLE CONNECTION CATHETER APPARATUS

(71) Applicant: YAIR MENDELS, MOZA ELIT (IL)

(72) Inventors: Yair Mendels, Moza Ilit (IL); Matan Shamir, Jerusalem (IL)

(21) Appl. No.: 14/385,949

(22) PCT Filed: Mar. 8, 2013
(86) PCT No.: PCT/US2013/029848

§ 371 Date: Sep. 17, 2014

Related U.S. Application Data
(60) Provisional application No. 61/613,643, filed on Mar. 21, 2012.

(51) Int. Cl. A61M 25/00; A61M 39/10; A61M 1/36; A61M 39/22

(52) U.S. Cl. CPC A61M 25/0097; A61M 39/223; A61M 25/0028; A61M 39/105; A61M 1/3653; A61M 2039/229
USPC 604/28; 604/246; 604/248; 604/508

(57) ABSTRACT

Apparatus is provided for a reversible connection catheter comprising in combination a hub and a valve housing portion. The hub is composed of a tapered molded body having a narrow tapered end and an enlarged other end. The narrow tapered end has integrated therein a double lumen catheter projecting therefrom. Two bores extend through the hub each communicating with a distinctive lumen in the catheter. The valve housing portion is composed of a distal segment with two spaced through first openings, a proximal segment with two spaced through second openings and a resilient gasket portion with two spaced first female luers mounted between the proximal and distal segments. The valve housing further has a third opening in said proximal segment positioned between the first and second openings and a second female luer on the resilient gasket portion in alignment with said third opening to serve as a flushing valve. A structural member operatively holds the hub and valve housing portion together. A stopcock may be interposed and held between the hub and the valve housing portion.

### CROSS REFERENCE TO RELATED APPLICATION

This application is related to and claims priority from U.S. Provisional Application No. 61/613,643 filed on Mar. 21, 2012, herein incorporated by reference in its entirety.

### BACKGROUND OF THE INVENTION

The invention relates to apparatus comprising a hub composed of a catheter hub portion in combination with a valve housing portion for terminating a catheter in a secure and sterile manner.

Various systems and methods are possible for a reversible connection catheter assembly or apparatus and, more particularly, a hub may provide a sterile and convenient reversible connection of a hemodialysis apparatus to multiple catheters located in a human patient. A common feature of catheters is that they are often used for long term treatment of “outpatient” patients wherein the patient comes to the hospital for treatment, such as chemotherapy, dialysis, TPN, Chronic pain relief etc. Catheters remain in the patient when he leaves the hospital and when he/she is involved in normal activity. Catheters often include multiple connectors for multiple fluid inlets and outlets to the patient's body. In current state of the art catheters, these inlets and outlets protrude from the body even when they are not in use and may be unsightly, may inhibit normal activity of the patient, and may become contaminated thereby allowing infection into the patient's body.

### SUMMARY OF THE INVENTION

The invention provides a reversible connection catheter assembly, device or apparatus and, more particularly, including a device or apparatus comprising a hub composed of a catheter hub portion in combination with a valve housing portion that are securely attachable and detachable for providing a sterile and convenient reversible connection of a hemodialysis apparatus to multiple catheters located in a human patient that is not unsightly, will not inhibit normal activity of the patient, and will not readily become contaminated or allow infection into the patient's body. Further, the valve housing portion, which is most likely to become unusable, can be readily detached or removed from the catheter hub portion and a new valve housing portion can be readily attached to the catheter hub portion, even without disturbing the position and functioning of the hub relative to a patient.

### BRIEF DESCRIPTION OF THE DRAWINGS

FIGS. 1a-c show a first preferred embodiment of the extension-less catheter apparatus including a hub in a top exploded view (FIG. 1a) and in a bottom exploded view (FIG. 1b), and the hub fastening mechanism in a top exploded view (FIG. 1c);

FIG. 2 shows the catheter hub in an assembled isometric view;

FIGS. 3a-3c show a partial cross-sectional cut through a lumen of the assembled hub without a resilient flexible member (FIG. 3a), with the resilient flexible member (FIG. 3b) and with an external connector (FIG. 3c);

FIG. 4 shows a cross-sectional side view of the hub with elongated thread screw of the hub fastening mechanism bent outward for the bolt to be positioned within the molded catheter portion;

FIG. 5 shows a screw driver and connector holder for attaching an external connector to the hub;

FIGS. 6a and 6b show an external connector in a top isometric view (FIG. 6a) and in in a bottom isometric view (FIG. 6b);

FIGS. 7a-c show the process for attaching a connector to the hub, where the connector is grasped by the connector holder (FIG. 7a), inserted partially into the hub (FIG. 7b) and inserted fully and fastened to the hub (FIG. 7c);

FIGS. 8a and 8b show a cross-sectional front view (FIG. 8a) and side view (FIG. 8b) of the connector attached to the hub of FIG. 7c.

FIGS. 9a-b show another embodiment of an external connector, consisting of a sealing cover for hub, in a top isometric view (FIG. 9a) and in a bottom isometric view (FIG. 9b);

FIGS. 10a-b show a top isometric view of a priming connector (FIG. 1a) and a bottom isometric view;

FIGS. 11a-b show a top view of the hub with a priming connector attached thereto (FIG. 11a) and a front cross-sectional view (FIG. 11b) taken along line A-A of FIG. 11a;

FIGS. 12a-b show an alternative embodiment of a priming connector in a top isometric view (FIG. 12a) and in a bottom isometric view (FIG. 12b);

FIGS. 13a-b show an exemplary technique for attaching a priming connector to the hub, with the distal tips of a pincer like gripping tool disposed within gripper receivers (FIG. 13a), and priming connector attached to the hub (FIG. 13b);

FIGS. 14a-b show a valve housing cover and connector cover, for use with priming connector, in an exploded view (FIG. 14a) and in an assembled view (FIG. 14b);

FIGS. 15a-b show a second embodiment of an external connector comprising extension fluid lines along with a valve housing cover separated from the hub (FIG. 15a) and assembled with the hub (FIG. 15b);

FIGS. 16a-b show a second embodiment of an external connector sealing cover along with a safety enclosure in an exploded view and an assembled view, respectively;

FIG. 17 shows a second embodiment of the valve housing portion of a catheter hub;

FIGS. 18a-c show a third embodiment of an external connector comprising extension fluid lines in a top isometric view (FIG. 18a), in a bottom isometric view (FIG. 18b) and in a cross-sectional view (FIG. 18c);

FIGS. 19a-b show a third embodiment of an external connector, consisting of a sealing cover for hub, in a top isometric view (FIG. 9a) and in a bottom isometric view (FIG. 9b);

FIGS. 20a-c show another embodiment of the catheter hub with the proximal segment separate from (FIG. 20a) and assembled with (FIG. 20b) the rest of the hub, and an external connector comprising extension fluid lines (FIG. 20c);

FIG. 21 shows an isometric view of a first embodiment of proximal and distal plugs;

FIG. 22 shows an isometric view of a second embodiment of proximal and distal plugs;

FIGS. 23a-b show the proximal and distal plugs in the process of attaching a connector to the catheter hub (FIG. 23a) and when attached (FIG. 23b);

FIGS. 24a-d show side views of the process of removing a connector from the hub using proximal and distal plugs;

FIGS. 25a-b show an alternative embodiment of a connector for the second embodiment of the catheter hub in a top isometric view (FIG. 25a) and bottom isometric view (FIG. 25b);

FIGS. 26a-c show a third embodiment of the catheter hub in an exploded bottom view (FIG. 26a), exploded top view (FIG. 26b) and assembled isometric view (FIG. 26c);

FIG. 27 shows the assembled hub of the third embodiment in an assembled view, with a partial cross-sectional cut along one lumen;

FIGS. 28a-b show an external connector with extension lines separate from the hub (FIG. 28a) and assembled with the hub (FIG. 28b);

FIGS. 29a-b show a front cross-sectional view of the hub taken transversely through the lumens of the hub and connector, prior to joining the connector to the hub (FIG. 29a) and when connected (FIG. 29b);

FIGS. 30a-c show an embodiment of a flushing connector in a top isometric view (FIG. 30a), a bottom isometric view (FIG. 30b) and in a cross-sectional front view assembled with the hub (FIG. 30c);

FIGS. 31a-c show a fourth embodiment of the catheter hub with a stopcock mechanism in an exploded bottom view (FIG. 31a), exploded top view (FIG. 31b) and assembled isometric view (FIG. 31c);

FIGS. 32a-b show a cross-sectional front view of the fourth embodiment of the hub, assembled with the external connector of FIGS. 28a-b, with the stopcock in an open position (FIG. 32a) and a closed position for flushing (FIG. 32b);

FIGS. 33a-b show a fifth embodiment of the catheter hub in an exploded bottom view (FIG. 33a), an exploded top view (FIG. 33b) and an assembled view (FIG. 33c);

FIGS. 34a-b show a cross-sectional front view of the fifth embodiment of the hub with extension lines prior to connecting with the hub (FIG. 34a) and when connected with the hub (FIG. 34b);

FIGS. 35a-c show a sixth embodiment of the catheter hub with a stopcock mechanism in an exploded bottom view (FIG. 35a), exploded top view (FIG. 35b) and assembled isometric view (FIG. 5); and,

FIGS. 36a-b show a cross-sectional front view of the sixth embodiment of the hub, assembled with the external connectors of FIGS. 33a-c, with the stopcock in an open position (FIG. 36a) and a closed position for flushing (FIG. 36b).

### DETAILED DESCRIPTION OF THE PREFERRED EMBODIMENTS

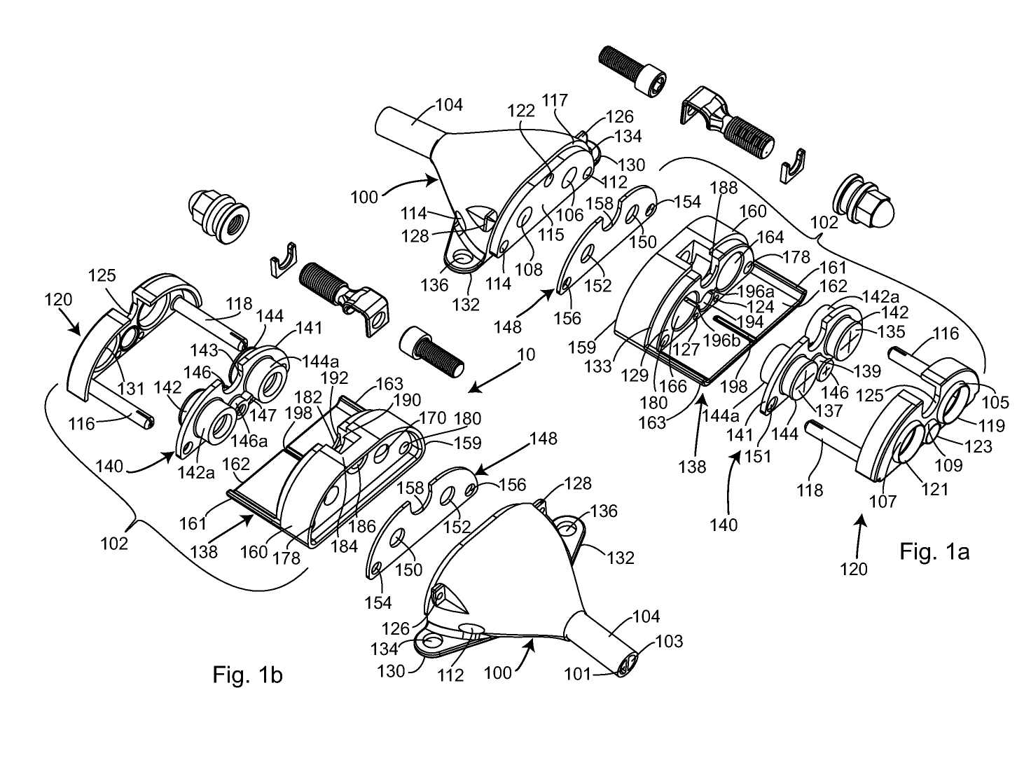

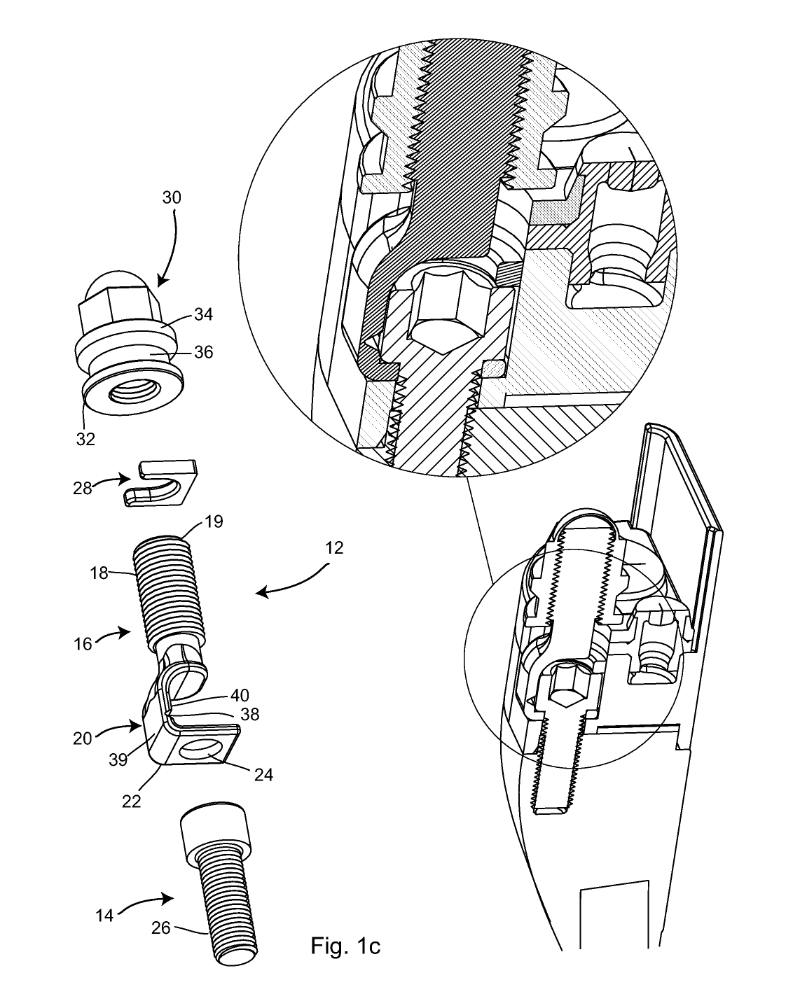

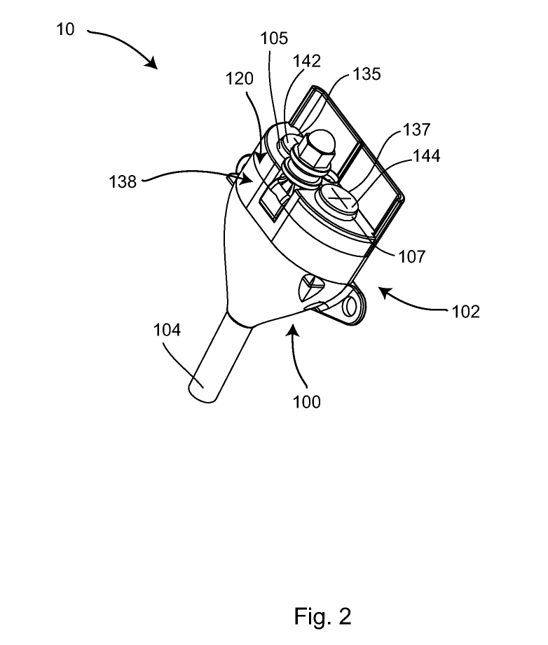

A first preferred embodiment of the extension-less catheter device comprising a hub (10) is shown in FIG. 1a in a top exploded view and in FIG. 1b in a bottom exploded view, and a fastening mechanism (12) is shown in a top exploded view in FIG. 1c. FIG. 2 shows device (10) in an assembled isometric view. Referring to FIGS. 1a-b and FIG. 2, device (10) comprises a molded catheter hub portion (100) and a valve housing portion (102). Molded catheter portion (100) is preferably insert molded with catheter tube (104), but may be manufactured as an independent component and joined to tube (104) in any suitable known way. Tube (104) is preferably manufactured from a flexible biocompatible plastic material.

Lumens (106), (108) extend through molded catheter portion (100) from proximal side (115) and are aligned with lumens (101), (103) in tube (104). Channels (112), (114) are situated at opposing transverse ends of proximal side (115) of molded catheter portion (100) for receiving respective guide pins (116), (118) of proximal segment (120) of valve housing portion (102), described herein below. Threaded cavity (122), for receiving bolt (14) (see FIG. 1c) of hub fastening mechanism (12) is situated at the front edge of the proximal side (115) of molded catheter portion (100). Ledges (126), (128), for fastening external connectors (not shown) to device (10) extend transversely from the front of molded catheter portion (100). Suturing ears (130), (132), having holes (134), (136) for attaching device (10) to a patient (not shown), extend transversely from the rear wall of molded catheter portion (100).

Valve housing portion (102) comprises a distal segment (138) and a proximal segment (120). A resilient flexible member (140) is disposed between distal segment (138) and proximal segment (120), and a gasket layer (148) is positioned between distal segment (138) and molded catheter portion (100). The components of valve housing portion (102) will now be described in greater detail.

Gasket layer (148) is preferably manufactured from a suitable elastic material and is shaped such that its edges align with the edges of proximal side (115) of molded catheter portion (100). Openings (150), (152) are aligned with lumens (106), (108) respectively, of molded catheter portion (100). Similarly, openings (154), (156) are aligned with channels (112), (114) respectively, of molded catheter portion (100). A u-shaped section (158) is cut-out from the front edge of gasket layer (148). The width of u-shaped section (158) is larger than the diameter of cavity (122) of molded catheter portion (100) such that u-shaped section (158) is wider than cavity (122). In one embodiment the proximal and distal surfaces of gasket layer (148) are essentially smooth, as shown in the figures. In an alternative embodiment (not shown) proximal and distal surfaces of gasket layer (148) comprise an array of dimples for storing and slowly releasing anticoagulant and/or antimicrobial agents.

Distal segment (138) is preferably manufactured from a high durometer plastic material, and includes a body (160) and a backboard (162). Body (160) includes two lumens (164), (166) respectively aligned with lumens (106), (108) of molded catheter portion (100).

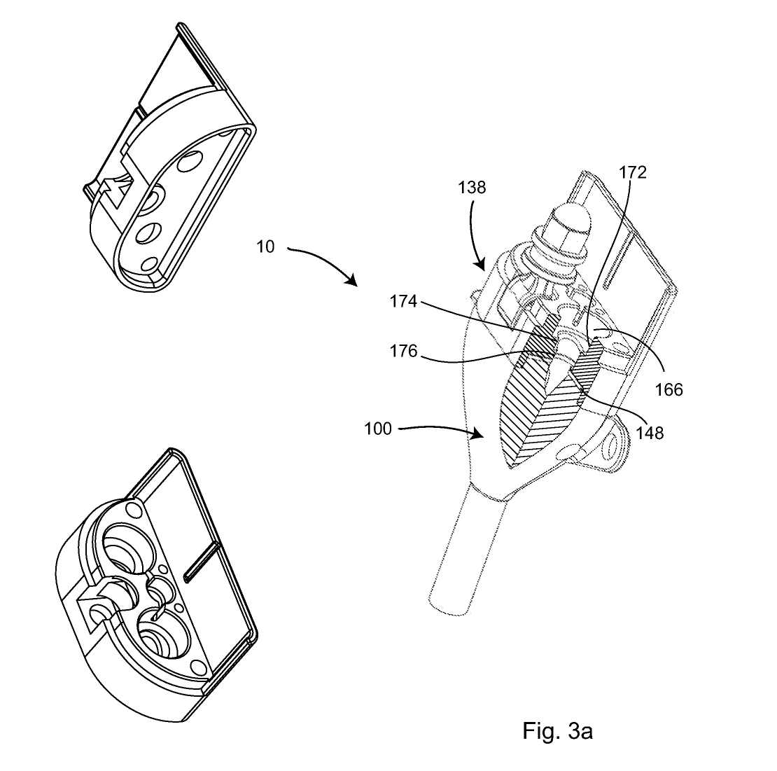

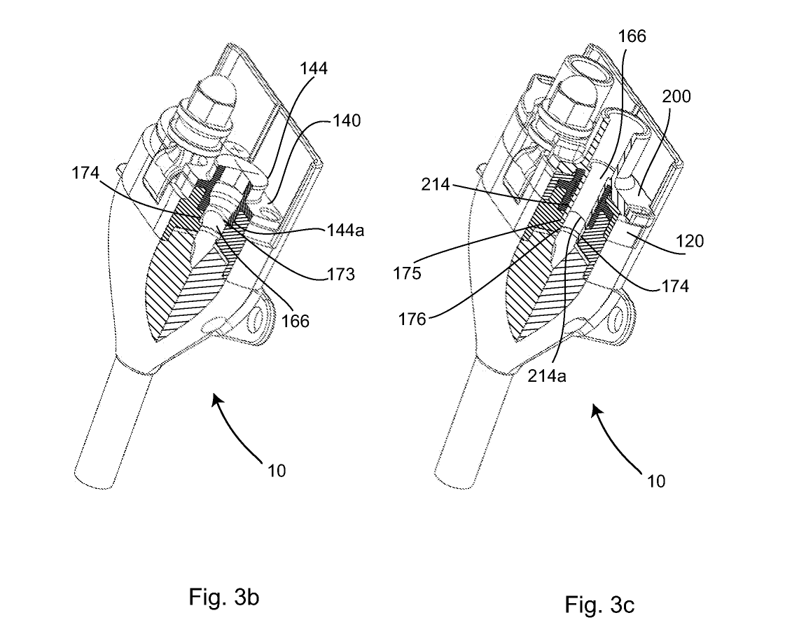

FIGS. 3a-c show an isometric view of the assembled device (10). FIG. 3a shows device (10) with only molded catheter portion (100), gasket layer (148) and distal segment (138). FIG. 3b shows device (10) of FIG. 3a with flexible gasket portion (140). FIG. 3c shows device (10) of FIG. 3b with proximal segment (120) and external connector (200). A partial cross-sectional cut is taken longitudinally through lumen (166) in each of FIGS. 3a-c. As best seen in FIG. 3a, the inner diameter of lumen (166) (as well as lumen (164), see FIG. 1a) decreases from the proximal to the distal direction. The change in diameter is shown in a stepped arrangement such that the proximal diameter (172) of lumen (166) is larger than the middle diameter (174) and middle diameter (174) is larger than distal diameter (176). FIG. 3b shows distal portion (144a) of valve (144) positioned within lumen (166) extending to the proximal rim (173) of middle diameter (174). FIG. 3c shows the cannula (214) of an external connector (200) positioned within lumen (166). The distal tip (214a) of cannula (214) terminates at a diagonal and rests on the rim (175) of distal diameter (176). Thus, the longitudinal length of middle and distal diameters (174), (176) are not equal, in order to accommodate the distal tip (214a) of cannula (214). Hence, in FIG. 3c, a smooth transition from cannula (214) to distal diameter (176) to enable an even surface along which fluid may flow through the lumens of device (10) when both the resilient flexible member and an external connector are assembled therewith.

Referring again to FIGS. 1a-b, channels (178), (180) align with openings (154), (156) of gasket layer (148) and with channels (112), (114) of molded catheter portion (100).

A U-shaped section (182) is cut out from a portion of the front (184) of distal segment (138). A longitudinal opening (186) in the remaining, uncut portion of front (184) of distal segment (138) is aligned with cavity (122) of molded catheter portion (100). Distal and proximal locking grooves (188), (190) are situated within u-shaped section (182) of distal segment (138), spaced apart from each other and disposed between longitudinal opening (186) and proximal edge (192) of u-shaped section (182), for receiving components of hub fastening mechanism (12) as described herein below.

A cavity (194) is situated between channels (164), (166). Slots (196a), (196b) are filled by sealing walls (145), (147) of resilient flexible member (140), as described herein below, to prevent fluid communication between cavity (194) and channels (164), (166).

A groove (198) runs longitudinally along the center of backboard (162) of distal segment (138), for guiding external connectors to device (10). Distal edges (161), (163) of backboard (162) are curved forward for further guiding external connectors to device (10).

When assembled, the lower edge (159) of body (160) of distal segment (138) is positioned on rim (117) extending around the perimeter of the raised proximal side (115) of molded catheter portion (100). Gasket layer (148) is situated between distal segment (138) and molded catheter portion (100) such that valve housing portion (102) is connected in a sealed manner to molded catheter portion (100).

Resilient flexible member (140) is preferably manufactured from a low durometer material, and comprises a layer (141) having essentially the same outer contour as that of gasket (148), including a u-shaped cut-out section (143). Valves (142), (144) and one-way flushing (or, priming) valve (146) extend both distally and proximally from layer (141). The distal portions (142a), (144a), (146a) of valves (142), (144), (146) are disposed within lumens (164), (166) and cavity (194), respectively, of distal segment (138). Sealing walls (145), (147) join distal portion (146a) of flushing valve (146) with distal portions (142a), (144a) of valves (142), (144), and are disposed within slots (196a), (196b) of distal segment (138) of valve housing portion (102), for preventing fluid communication between lumens (164), (166). The proximal end of each of valves (142), (144), (146) comprises a slitted surface which is normally closed, for disinfection swabbing. Openings (149), (151) at transverse ends of layer (141) align with channels (178), (180), with openings (154), (156) and with channels (112), (114).

Proximal segment (120) of valve housing portion (102) is preferably manufactured from a strong plastic material, and maintains resilient flexible member (140) disposed within lumens (164), (166) and cavity (194), respectively, of distal segment (138). Guiding pins (116), (118) are passed through openings (149), (151), channels (178), (180), openings (154), (156) and channels (112), (114), respectively. Distal tips of pins (116), (118) are slotted and comprise a slightly wider diameter than the rest of the length of pins (116), (118) for providing a snug fit within channels (112), (114) and preventing pins (116), (118) from falling out of channels (112), (114). Referring to FIG. 1a and FIG. 2, when assembled, rings (105), (107) extending proximally from openings (119), (121) are disposed around valves (142), (144) respectively, between the wide diameter heads (135), (137) and layer (141). Rings (105), (107), (109) provide support to wide diameter heads (135), (137), (139), respectively, by preventing valves (142), (144), (146) from collapsing during use of catheter device (10).

U-shaped cut-out section (125) of proximal segment (120) is aligned with u-shaped section (143) of resilient flexible member (140) and with u-shaped section (182) of distal segment (138). The distal edge (131) of proximal segment (120) is positioned on rim (133) extending around the perimeter of the raised proximal side (129) of distal segment (138).

With reference to FIG. 1c, hub fastening mechanism (12) is utilized for connecting distal segment (138) of valve housing portion (102) with molded catheter portion (100), as well as for affixing external connectors to device (10). Hub fastening mechanism (12) comprises a bolt (14) and coupling element (16), which are preferably manufactured from a strong plastic material or metal. Coupling element (16) comprises a proximal elongated threaded screw (18) and a distal bolt holding portion (20). Bolt holding portion (20) is an L-shape (or, hook shape) bracket extending longitudinally from the distal end of threaded screw (18). The foot (22) of bolt holding portion (20) includes an opening (24) through which the elongated threaded member (26) of bolt (20) passes. Hub fastening mechanism (12) further comprises a u-shaped bolt locking element (28) having a smooth inner contour and a square outer contour, and a nut fastener (30) for threading onto the proximal end (19) of elongated threaded screw (18) and tightening an external connector thereto, as described herein below. Nut fastener (30) comprises a distal ring (32) and a proximal ring (34) spaced apart by a neck section (36) having a smaller outer diameter than distal and proximal rings (32), (34).

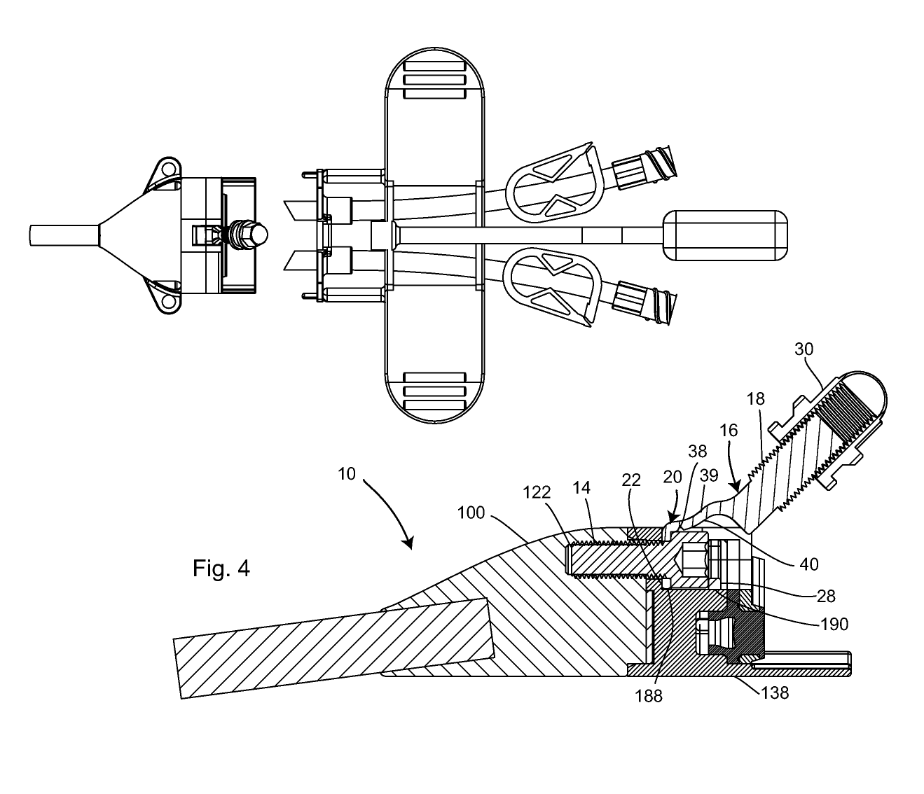

Referring to FIG. 4, showing a cross-sectional side view of device (10), in order to connect distal segment (138) of valve housing portion (102) with molded catheter portion (100) via hub fastening mechanism (12) foot portion (22) of coupling element (16) is first lodged in distal locking groove (188) of distal segment (138) such that the opening (24) (see FIG. 1c) is aligned with longitudinal opening (186) (see FIG. 1a). Elongated threaded screw (18) is then bent away from device (10) to allow insertion of bolt (14) through openings (24), (186) and into cavity (122) in molded catheter portion (100). A niche (38) runs along the inner side (40) of the longitudinal leg (39) of bolt holding portion (20) for preventing a fracture from occurring when bending threaded screw (18). Locking element (28) is lodged in proximal locking groove (190) with the open side facing frontward. Nut fastener (30) is threaded at the proximal end of elongated thread screw (16).

It may be desirable to separate valve housing portion (102) from molded catheter portion (100), for instance, when repair or replacement of one or more parts of valve housing portion (102) is required. In that case, a standard hexagon head screwdriver, or hex key, is used to unscrew bolt (14) from cavity (122). Bolt locking element (28) prevents bolt (14) from exiting longitudinal opening (186). Thus, while bolt (14) is unscrewed from cavity (122), bolt (14) remains coupled with distal segment (138), and valve housing portion (102) is separated from molded catheter portion (100).

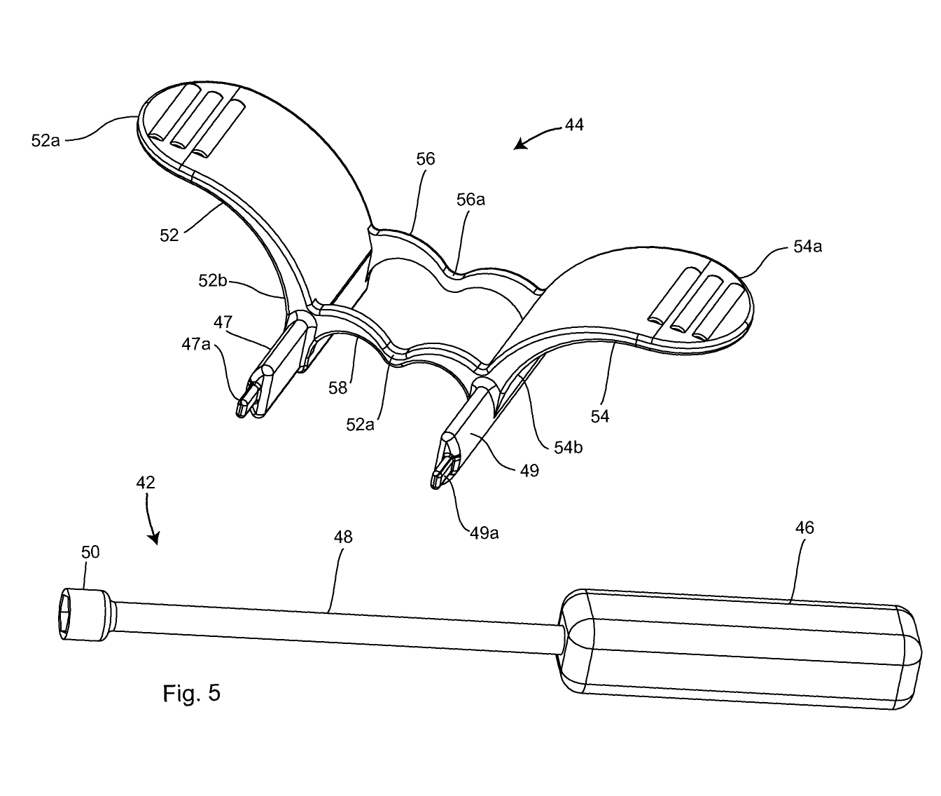

Referring to FIG. 5, the preferred tools for attaching an external connector to device (10) are shown: screw driver (42) and connector holder (44). Screw driver (42) is preferably manufactured from a strong plastic or metal, and includes a handle (46) and an elongated shaft (48) having an external hexagonal head (50).

Connector holder (44) is preferably manufactured from a strong plastic material and comprises wings (52), (54), each having an outer portion (52a), (54a) for grasping by the user, as well as an inner portion (52b), (54b) that are joined together by a proximal shoulder (56) and a distal shoulder (58). A recessed portion (56a), (56b), upon which shaft (48) is longitudinally positioned, is formed at essentially the center of each shoulder (56), (58). Longitudinal arms (47), (49) extend distally from inner portion (52b), (54b) of wings (52), (54). Fingers (47a), (49a) extend further distally from longitudinal arms (47), (49).

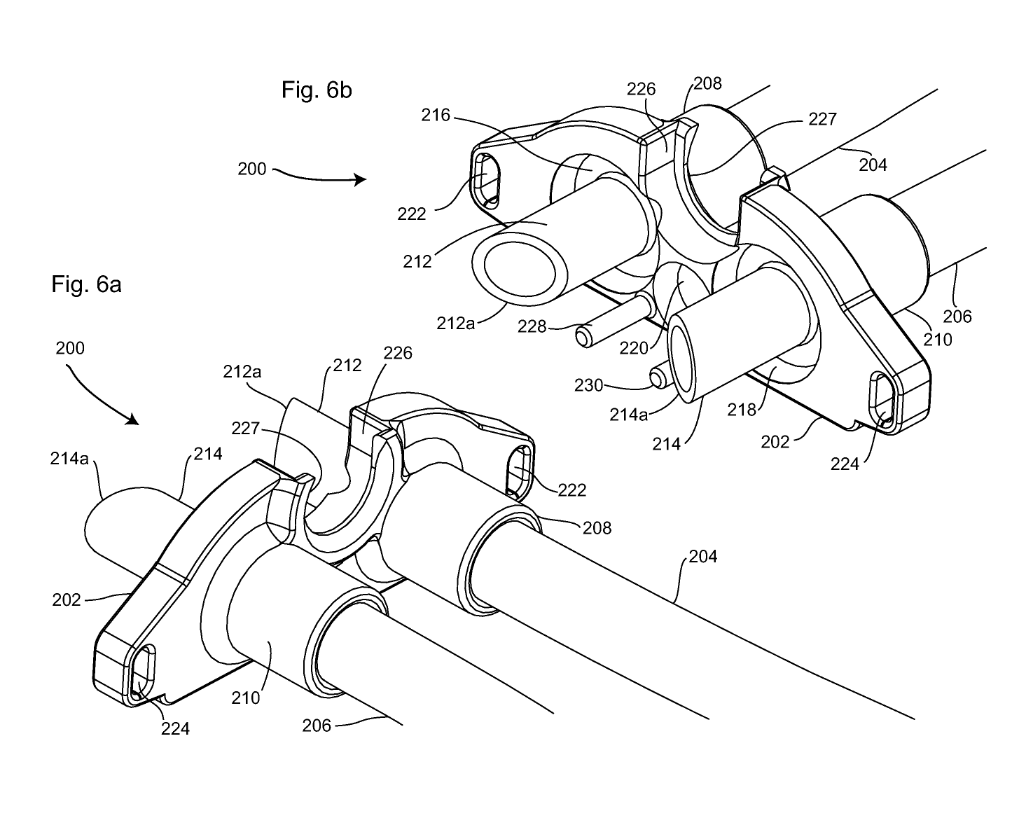

A first embodiment of an external connector (200) comprising extension fluid lines (204), (206) is shown in FIG. 6a in a top isometric view and in FIG. 6b in a bottom isometric view, comprising a main body (202) preferably manufactured from a high durometer strong plastic material. Fluid lines (204), (206) extend from the proximal side of main body (202) and are supported by extension holders (208), (210) extending from the proximal side of main body (202). Cannulas (212), (214) protrude from the distal side of main body (202), and comprise a distal tip (212a), (214a) that terminates at a sharp angle for protruding through the slits of valves (142), (144) in device (10) and extending through lumens (164), (166) (see FIG. 3c). As best seen in FIG. 6b, an annular depression (216), (218) surrounds each cannula (212), (214) for positioning over valves (142), (144), and an annular depression (220) is situated between annular depressions (216), (218) for positioning over valve (146). Apertures (222), (224) situated at the transverse ends of main body (202) are grasped by fingers (47a), (49a) of connector holder (44) (see FIG. 5) when attaching an external connector to device (10), as described further herein below. A central u-shaped section (226) is cut-out from the front of main body (202) for accommodating nut fastener (30). Proximal edge (227) of u-shaped section (226) comprises a u-shaped ring having a smaller than the rest of u-shaped section (226), for positioning at neck (36) of nut fastener (30), between rings (32), (34). Aligning pins (228), (230) extend distally from main body (202) for aligning connector (200) with device (10) by entering holes (124), (127) in proximal side (115) of distal segment (138), as described herein below.

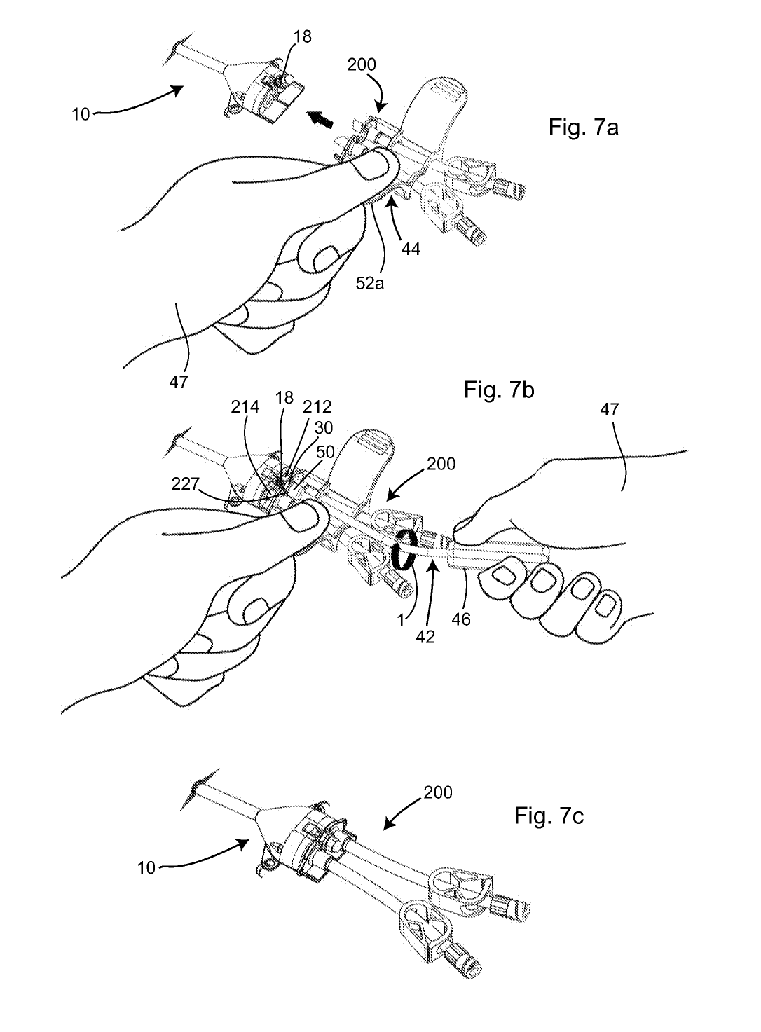

An exemplary technique for attaching connector (200) to device (10) using tools (42), (44) is shown in FIGS. 7a-c. FIG. 7a shows a user (47) grasping wing (52a) of connector holder (44), which is holding connector (200). Elongated threaded screw (18) is shown bent away from device (10) in preparation for attaching connector (200) to device (10).

In FIG. 7b cannulas (212), (214) are partially inserted within respective valves of device (10) and aligning pins (not shown) are partially inserted with respective holes (124), (127) (see FIG. 1a). User (47) is shown holding handle (46) of screw driver (42) with a second hand, and head (50) is mated with nut fastener (30). Elongated threaded screw (18) is returned to its normal (unbent) longitudinal position and proximal edge (227) of u-shaped section (226) of connector (200) is disposed within neck section (36) of nut fastener (30) between rings (32), (34) (see FIG. 1c) Arrow (1) is shown indicating the direction of rotation of screw driver (42) for further threading of nut fastener (30) along elongated threaded screw (18), thereby tightening connector (200) to device (10).

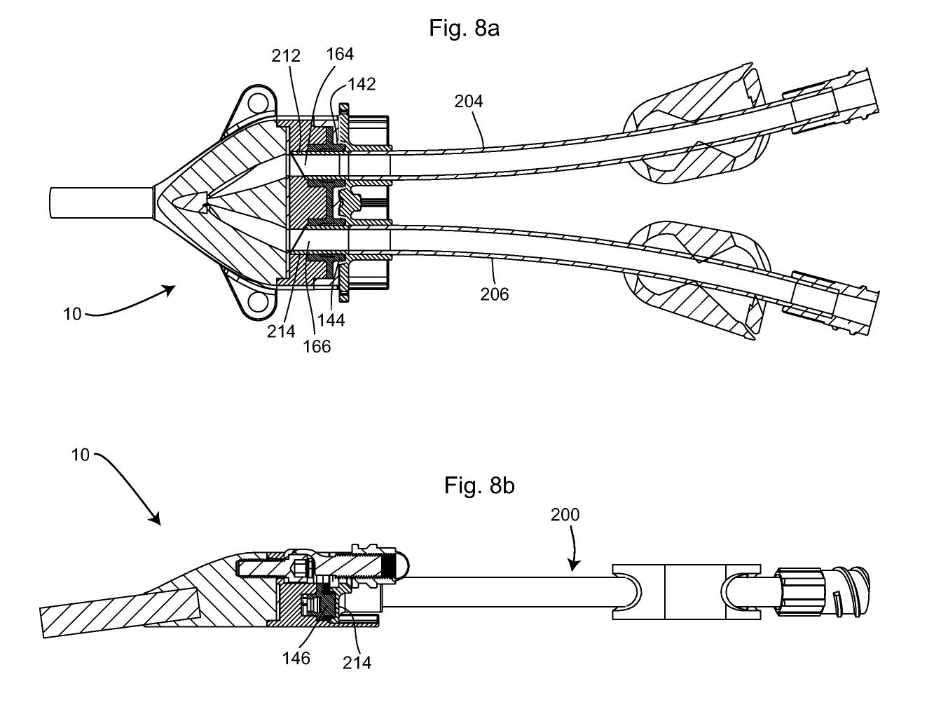

In FIG. 7c, connector (200) is shown attached to device (10) with the cannulas (212), (214) fully inserted within the valves (142), (146). FIG. 8a shows a cross-sectional front view of connector (200) attached to device (10) of FIG. 7c, taken along the lumens (164), (166), thus the components of the hub fastening mechanism are not shown. Cannulas (212), (214) are seen fully inserted within respective valves (142), (144). Thus, fluid lines (204), (206) are in fluid communication with lumens (164), (166) of device (10). All components of device (10) are sealingly pressed with each other to prevent leakage through the openings and/or spaces between the components. Unobstructed medication or blood flow through lumens (164), (166) during a dialysis procedure or any other medication infusion may be achieved.

FIG. 8b shows a cross-sectional side view of connector (200) attached to device (10). Annular depression (220) of connector (200) sealingly covers flushing valve (146) and prevents fluid communication with valves (142), (144).

In order to remove connector (200) from device (10), the essentially reverse procedure for securing connector (200) to hub (80), is performed. Screwdriver (42) is utilized to unscrew nut fastener (30). Nut fastener (30) and elongated threaded screw (18) are bent outward from device (10), and connector (200) is removed from device (10).

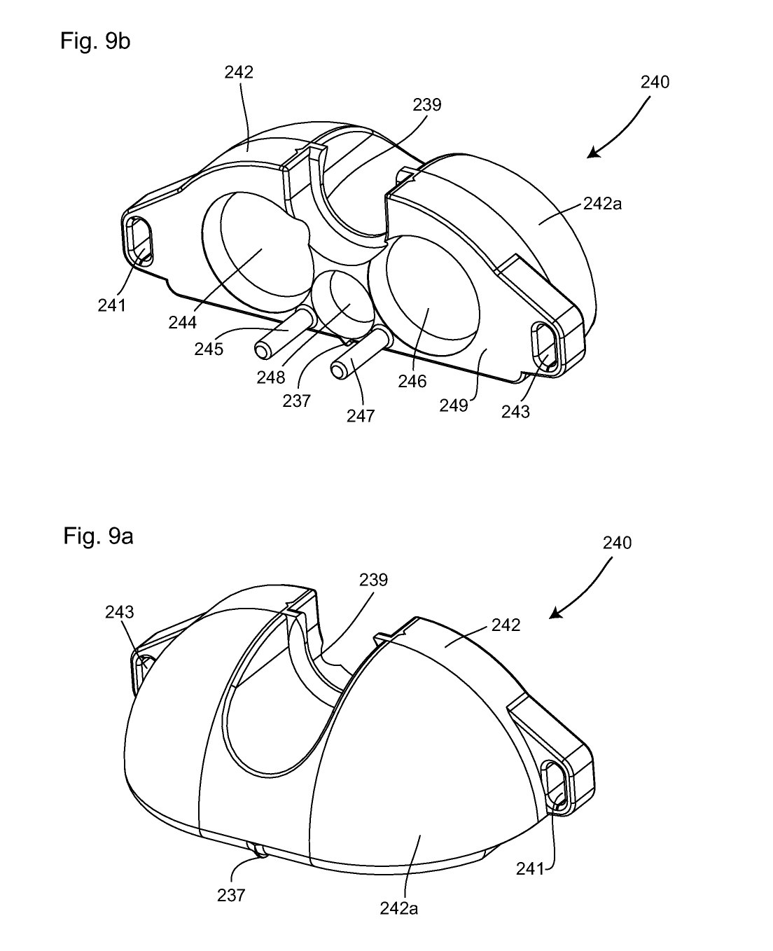

A first embodiment of an external connector (240) consisting of a sealing cover for device (10) is shown in FIG. 9a in a top isometric view and in FIG. 9b in a bottom isometric view, having the same essential features as that of connector (200), mutatis mutandis, and comprising a main body (242) having a rounded proximal front portion (242a), preferably manufactured from a high durometer strong plastic material. Annular depressions (244), (246), (248) in the distal side (249) of main body (242) serve as sealing covers for valves (142), (144), (146) for preventing fluid communication therethrough. Apertures (241), (243) at transverse ends of main body (242) are grasped by fingers (47a), (49a) of connector holder (44). Proximal edge (238) of u-shaped cut-out section (239) comprises a u-shaped ring having a smaller diameter than the rest of u-shaped section (239) for positioning at neck (36) of nut fastener (30), between rings (32), (34). Aligning pins (245), (247) extend distally from main body (242) for aligning connector (240) with device (10).

Also shown in FIGS. 9a-b is guiding rail (237) extending longitudinally along the back side of connector (240), for sliding along groove (198) along backboard (162) of distal segment (138), for guiding connectors (240) to device (10). A rail is present on connector (200), mutatis mutandis, although not seen in the figures.

The technique for attaching and removing sealing cover (240) to and from device (10) is essentially the same as that used for attaching and removing connector (200) to and from device (10), as described herein above, mutatis mutandis.

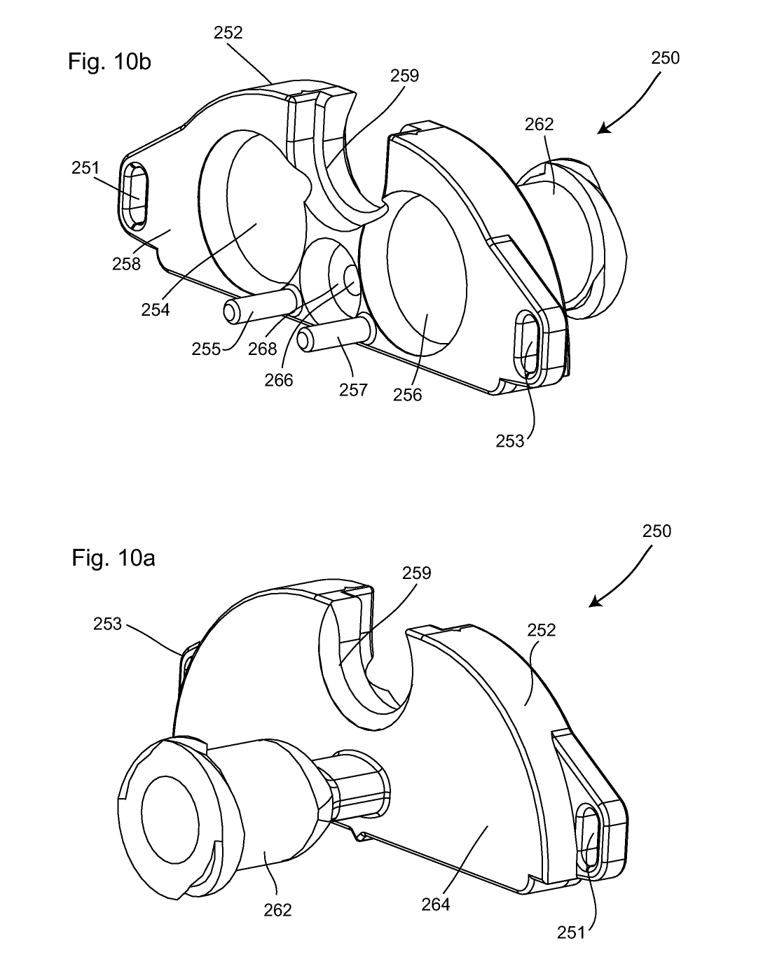

FIGS. 10a and 10b show a first embodiment of an external connector (250) consisting of a flushing connector for device (10). Connector (250) is shown in FIG. 10a in a top isometric view and in FIG. 10b in a bottom isometric view, having the same essential features as that of connector (200), mutatis mutandis, and comprising a main body (252) preferably manufactured from a high durometer strong plastic material. Annular depressions (254), (256), in the distal side (258) of main body (252) serve as sealing covers for valves (142), (144) for preventing fluid communication therethrough. Apertures (251), (253) are grasped by fingers (47a), (49a) of connector holder (44). Proximal edge (269) of u-shaped cut-out section (259) comprises a u-shaped ring having a smaller diameter than the rest of u-shaped section (259) for positioning at neck (36) of nut fastener (30), between rings (32), (34). Aligning pins (255), (257) extend distally from main body (242) for aligning connector (240) with device (10).

Flushing connector (250) further comprises a female luer lock (262) extending longitudinally from the proximal side (264) of main body (252), for introducing flushing (or, priming) fluid to the flushing valve (146) of device (10), through opening (266) in annular depression (268) in distal side (258) of main body (252).

The technique for attaching and removing flushing connector (250) to and from device (10) is essentially the same as that used for attaching and removing connector (200) to and from device (10), as described herein above, mutatis mutandis.

When attaching flushing connector (250) to device (10), flushing valve (146) is maintaining in its normally closed position until liquid pressure is applied through opening (266) of luer lock (262), as described herein below.

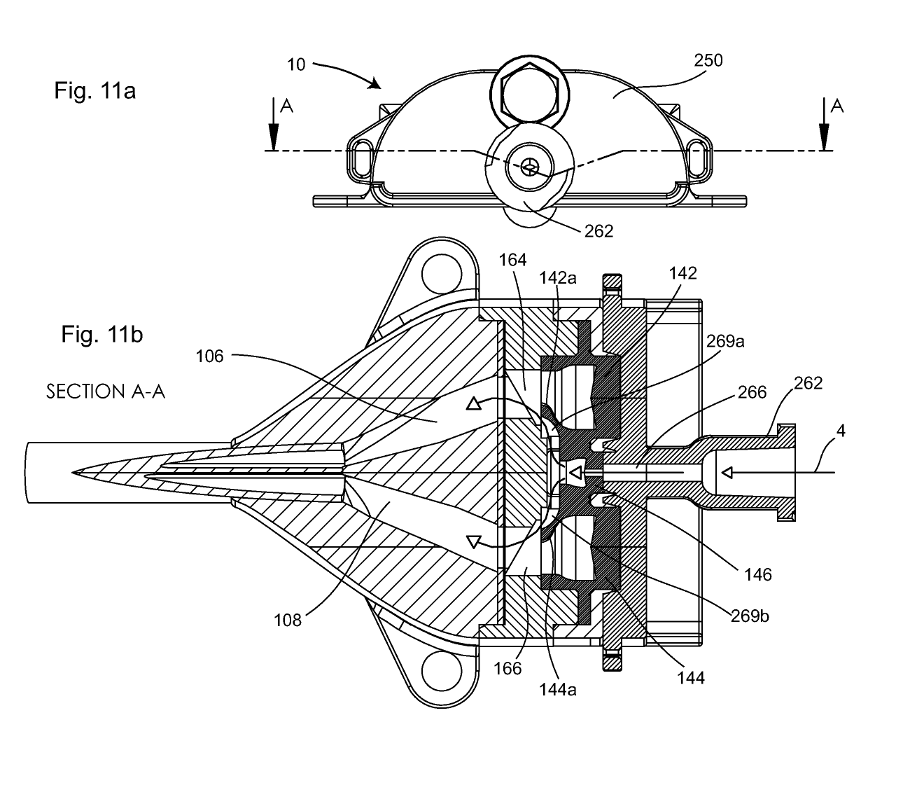

FIG. 11a shows a top view of device (10) with flushing connector (250) attached thereto. A cross-sectional cut line A-A is shown transversely along device (10), passing through female luer lock (262). A front cross-sectional view taken along A-A is shown in FIG. 11b. Priming fluid, indicated by arrow (4) is shown flowing through priming opening (266) of luer lock (262). When fluid pressure is applied to proximal flushing valve (146), valve slits separate, allowing priming fluid to pass through flushing valve (146). Fluid pressure forces the wall of distal portion (142a), (144a) of each of valves (142), (144) inward, toward the hollow of each valve (142), (144), thereby forming passageways (269a), (269b) for allowing the priming fluid to flow through distal portion of lumens (164), (166) and into lumens (106), (108) of molded catheter portion (100).

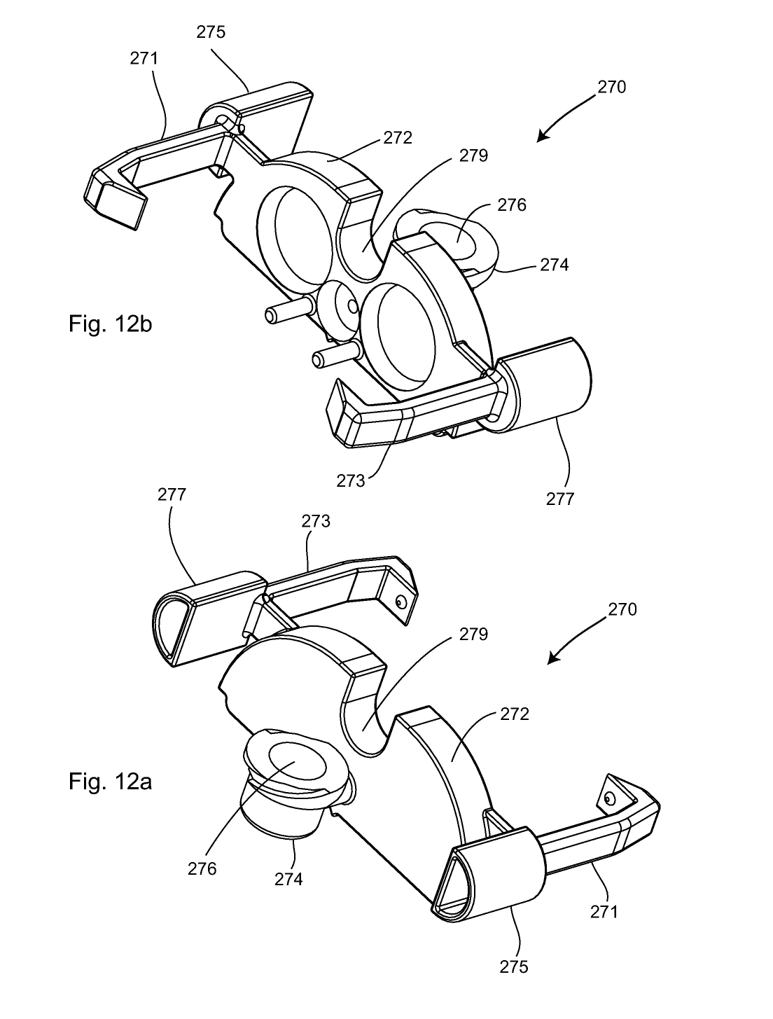

A second embodiment of a flushing connector (270) is shown in FIGS. 12a in a top isometric view and in FIG. 12b in a bottom isometric view. Flushing connector (270) includes all of the main features and elements of flushing connector (250), mutatis mutandis, with the following differences. Flushing connector (270) includes a main body (272) having an essentially smooth walled u-shaped cut-out section (279), as well as a female luer lock (274) oriented orthogonal to that of female luer lock (262) of flushing connector (250), such that opening (276) of female luer lock (262) faces toward the front of connector (270). Longitudinal elongated snapping hooks (271), (273) are flexibly attached at respective transverse ends of priming connector (270). Gripper receivers (275), (277) extend proximally from longitudinal hooks.

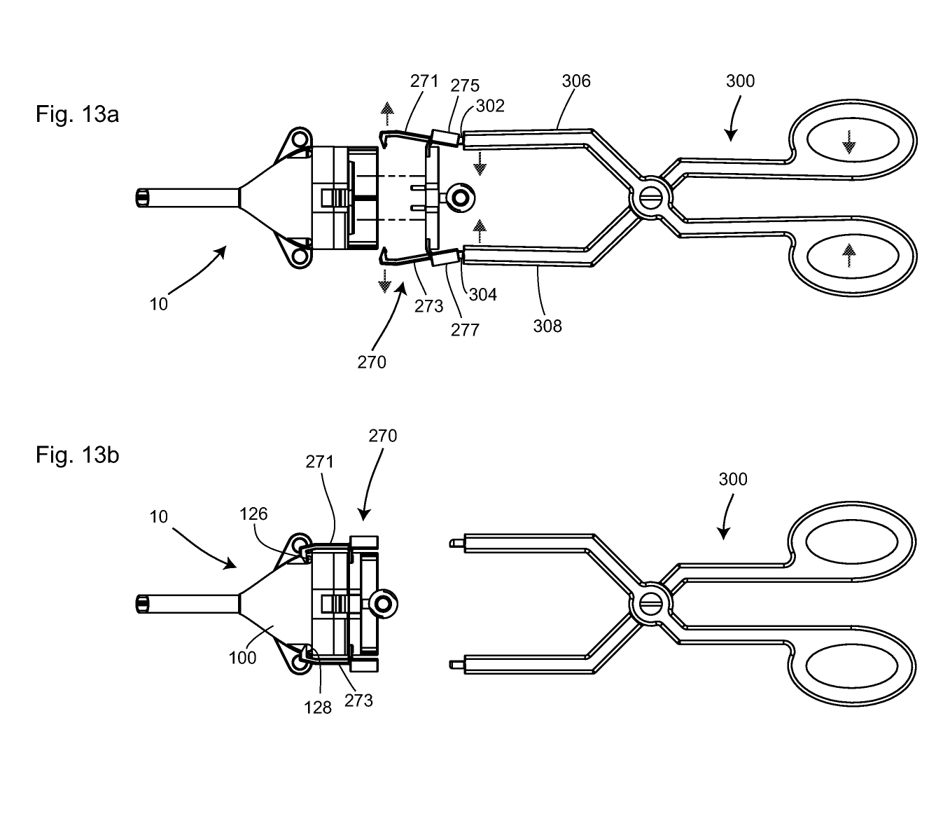

FIGS. 13a-b show an exemplary technique for attaching priming connector (270) to device (10) via a pincer-like gripping tool (300). In FIG. 13a, distal tips (302), (304) are disposed within gripper receivers (275), (277). Arms (306), (308) of tool (300) are pressed together, thereby separating snapping hooks (271), (273). FIG. 13b shows priming connector (270) attached to device (10), wherein the ends of snapping hooks (271), (273) are disposed beneath ledges (126), (128) of molded catheter portion (100).

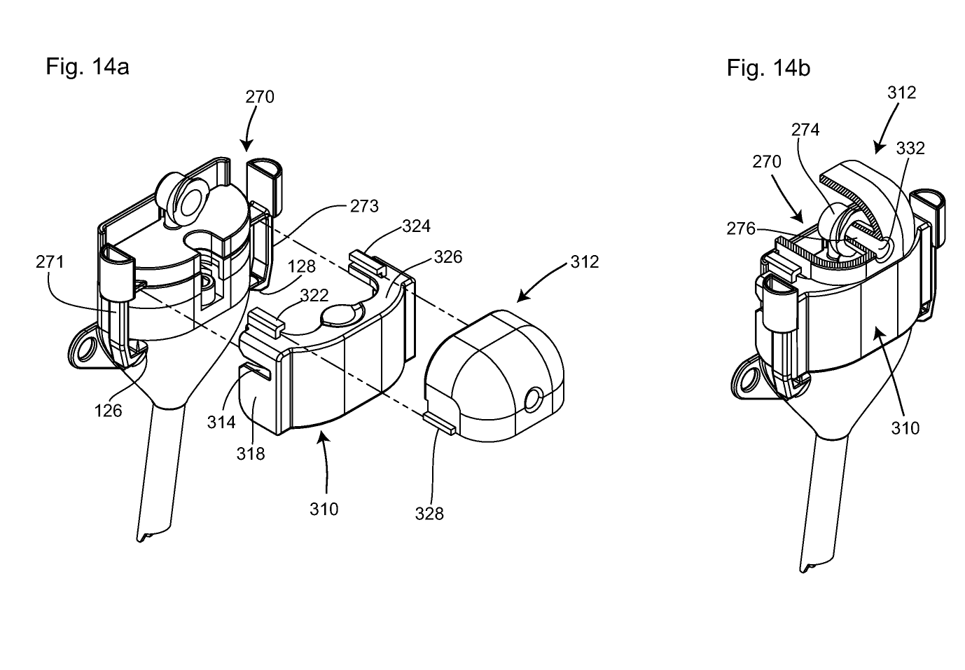

Referring to FIGS. 14a-b, a valve housing cover (310) and connector cover (312) are shown, for use with priming connector (270), in an exploded view (FIG. 14a) and in an assembled view (FIG. 14b), wherein connector cover (312) is partially sectioned. Valve housing cover (310) is essentially a transverse bracket having slots (314) at bracket arms (318) (only one slot and arm is shown) for sliding over transverse ends of priming connector (270), as indicated by dotted lines in the figure. This prevents snapping hooks (271), (273) from being unintentionally separated and dislodged from ledges (126), (128).

Rails (322), (324) are positioned along the proximal surface (326) of valve housing cover (310), for receiving corresponding guiding elements (328) positioned along the transverse sides of connector cover (312). FIG. 14b shows an integral plug (332) portion of connector cover (312) for plugging up opening (276) of luer lock (274).

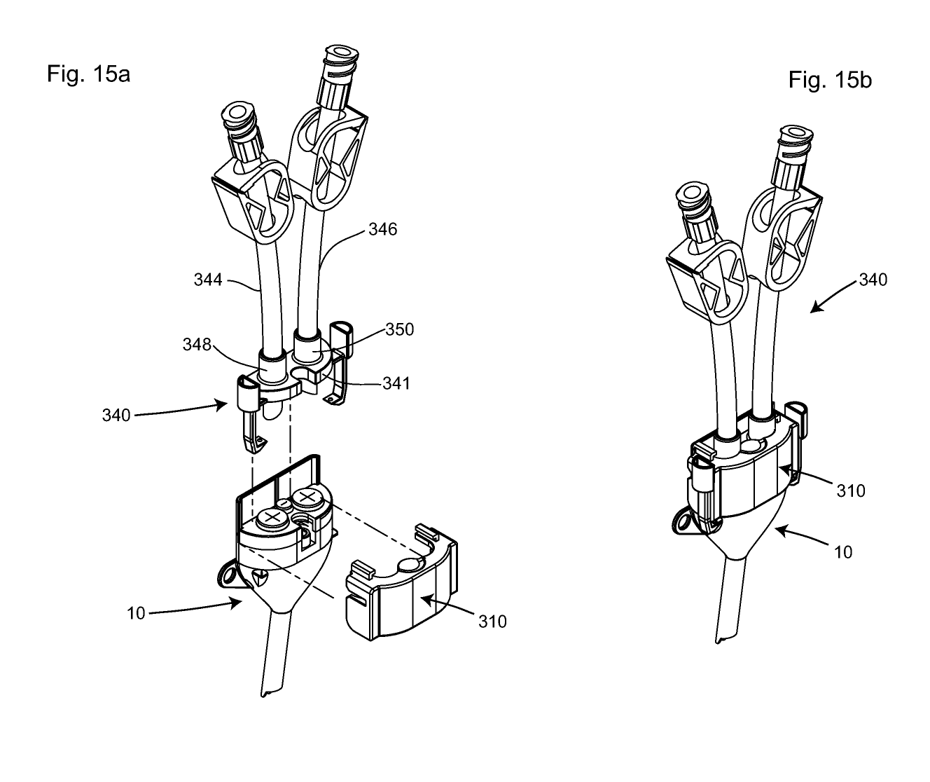

A second embodiment of an external connector (340) comprising extension fluid lines is shown in FIG. 15a along with a valve housing cover (310) separated from device (10). External connector (340) comprises the same essential components as that of flushing connector (270), mutatis mutandis, whereby fluid lines (344), (346) extend from the proximal side of main body (341) and are supported by extension holders (348), (350) extending from the proximal side of main body (341), instead of a female luer lock at the proximal side of main body (341), similar to fluid line extension connector (200). In FIG. 15b, connector (340) is shown attached to device (10) with valve housing cover (310) assembled with device (10).

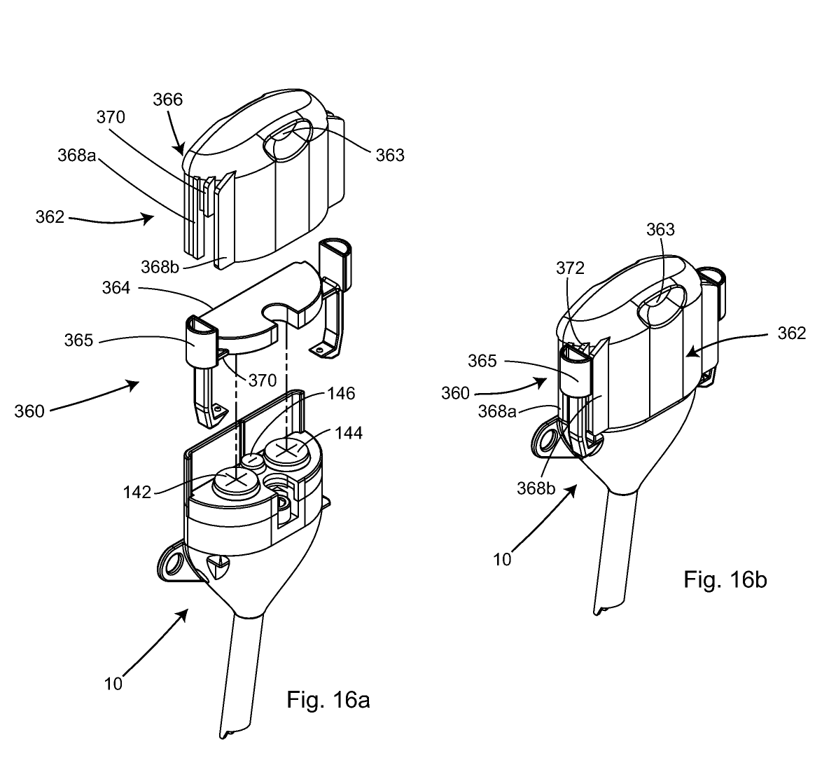

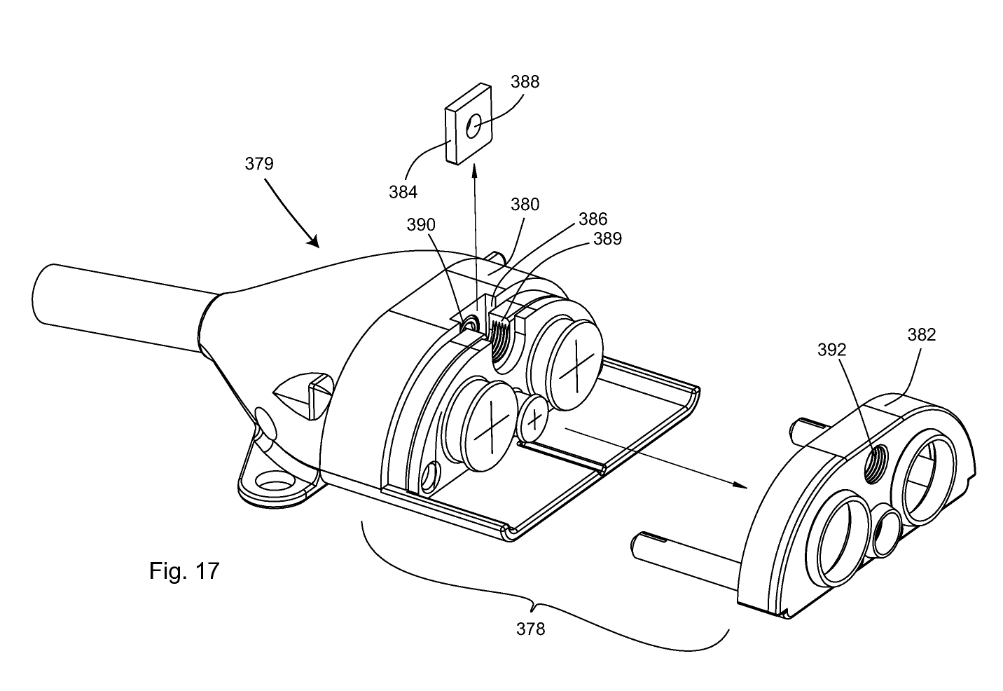

FIG. 16a shows a second embodiment of a sealing cover connector (360) with a safety enclosure (362) separated from device (10). Connector (360) comprises the same essential components as that of flushing connector (270), mutatis mutandis, whereby the main body (364) is sealed thereby sealing the proximal valves and the proximal priming valve of device (10), instead of a female luer lock extending therefrom, similar to the first embodiment of sealing cover (240).

When assembled, as shown in FIG. 16b, safety enclosure (362) prevents sealing cover (360) from being unintentionally removed from catheter device (10). Each transverse side (366) of safety enclosure (362) comprises spaced apart walls (368a), (368b) for sliding over transverse arms (370), and a longitudinal stopper (372) for preventing gripper receivers (365) from shifting inward. Enclosure (362) can also be used to cover flushing connector (270) when attached to device (10), whereby luer lock (274) is disposed within opening (363).

FIG. 17 shows a second embodiment valve housing portion (378) of a catheter hub (379), comprising alternative embodiments of distal segment (380), resilient flexible member (381), proximal segment (382) and bolt locking element (384), having all of the essential features of the first embodiment of device (10), mutatis mutandis, with the following differences. As seen in FIG. 17, distal segment (380) comprises a threaded u-shaped cut-out section (389) and a distal locking groove (386) in which bolt locking element (384) is disposed. Bolt locking element (384) comprises an essentially square piece having an opening (388) aligned with opening (390) in distal segment (380). Proximal segment (382) comprises a threaded opening (392) aligning with opening (390) in distal segment (380).

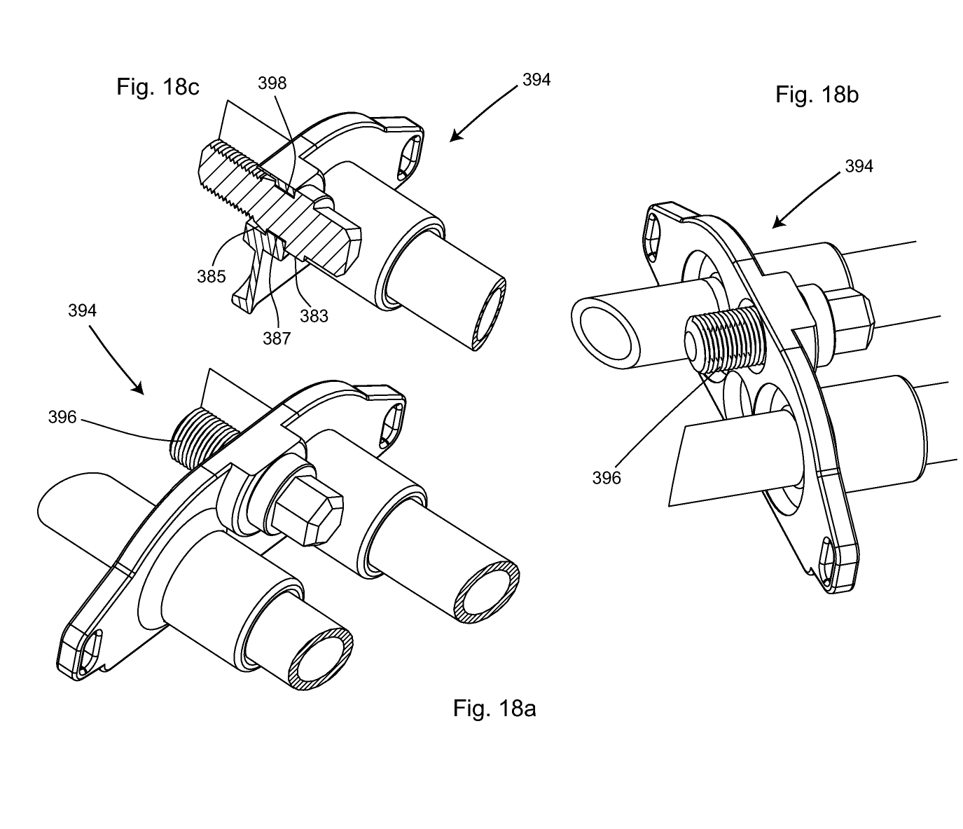

The hub fastening mechanism of hub (379) comprises bolt locking element (384) a distal bolt (not shown) for positioning in molded catheter portion (100) through opening (390), and a proximal bolt (see FIGS. 18a-c) for threading through openings (392), and with threaded u-shaped cut-out section (389).

A third embodiment of an external connector (394) comprising extension fluid lines is shown in FIGS. 18a-c, comprising the same essential features as that of connector (200), mutatis mutandis, with the following differences. As best seen in FIG. 18c, showing a cross-sectional isometric view of connector (394) taken longitudinally through bolt (396), bolt (396) is captive within opening (398). Bolt (396) comprises proximal and distal rings (383), (385) spaced apart by a small diameter neck (387) which is freely rotatable within opening (398).

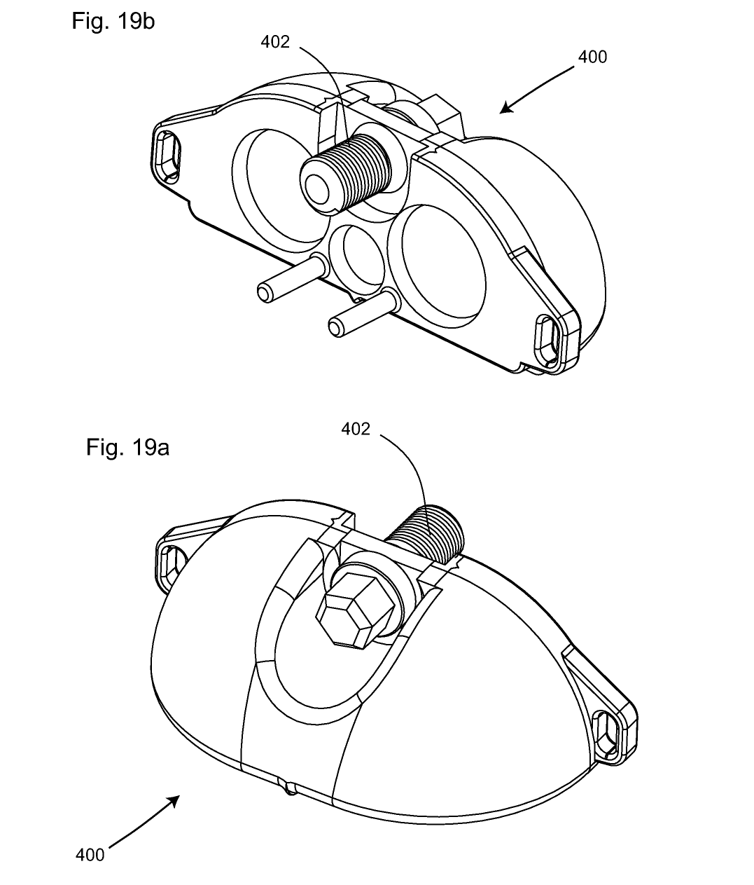

FIGS. 19a-b show a third embodiment of an external connector, consisting of a sealing cover (400) for a catheter hub, in a top isometric view (FIG. 19a) and in a bottom isometric view (FIG. 19b), comprising the same essential features as that of connector (240), mutatis mutandis, with the following differences. Connector (400) comprises a bolt (402) captive within opening (not seen), for threading with a catheter hub as described herein above with reference to connector (394).

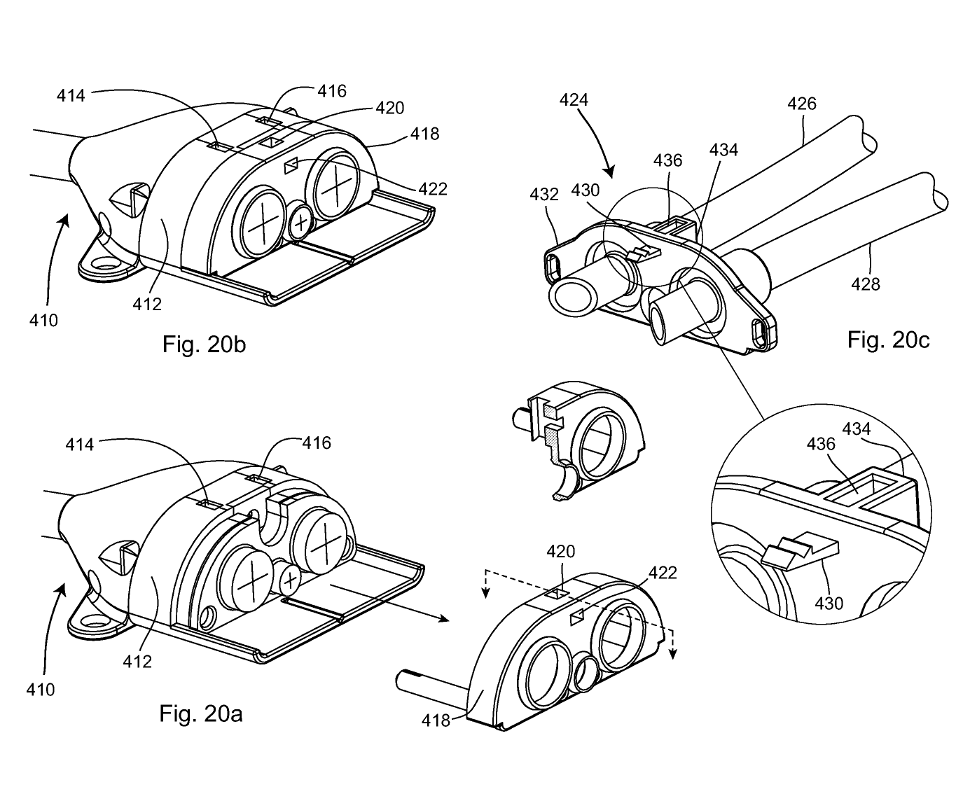

Referring to FIGS. 20a-c, a third embodiment of a catheter hub (410) is shown comprising the same essential components as the previous embodiments, mutatis mutandis, with the following differences. Two apertures (414), (416) are spaced apart from each other at the front of distal segment (412). Proximal segment (418) comprises a front aperture (420) and a proximal aperture (422). FIG. 20c shows an embodiment of an external connector (424) for attaching to hub (410), comprising extension fluid lines (426), (428) and having a proximal protrusion (434) extending from the proximal side of the main body (432) of connector (424) comprising a front facing cavity (436), and a double toothed tab (430) extending longitudinally from the distal side of main body (432).

Connector (424) is assembled with hub (410) via proximal male plug (440) and distal female plug (442) shown in FIG. 21. Referring also to FIGS. 20a-c, Proximal plug (440) comprises one transverse prong (444) for inserting into cavity (436) of proximal protrusion (434) in connector (424), and two longitudinal prongs (446), (448) for insertion into corresponding longitudinal sockets (450), (452) within distal plug (442). The tips of longitudinal prongs (446), (448) comprise projections (454) for preventing inadvertent removal from distal plug (442) when prongs (446), (448) are fully inserted into sockets (450), (452), similar to a side release buckle. Distal plug (442) comprises two transverse prongs (456), (458) for insertion into front apertures (414), (416) in distal segment (412), and a transverse locking wedge (460) for insertion into front aperture (420) of proximal segment (418). Transverse sides (461), (462) of plugs (440), (442) comprise anti-slip surfaces consisting of ribs (457), (459) for enabling the user to grasp easily.

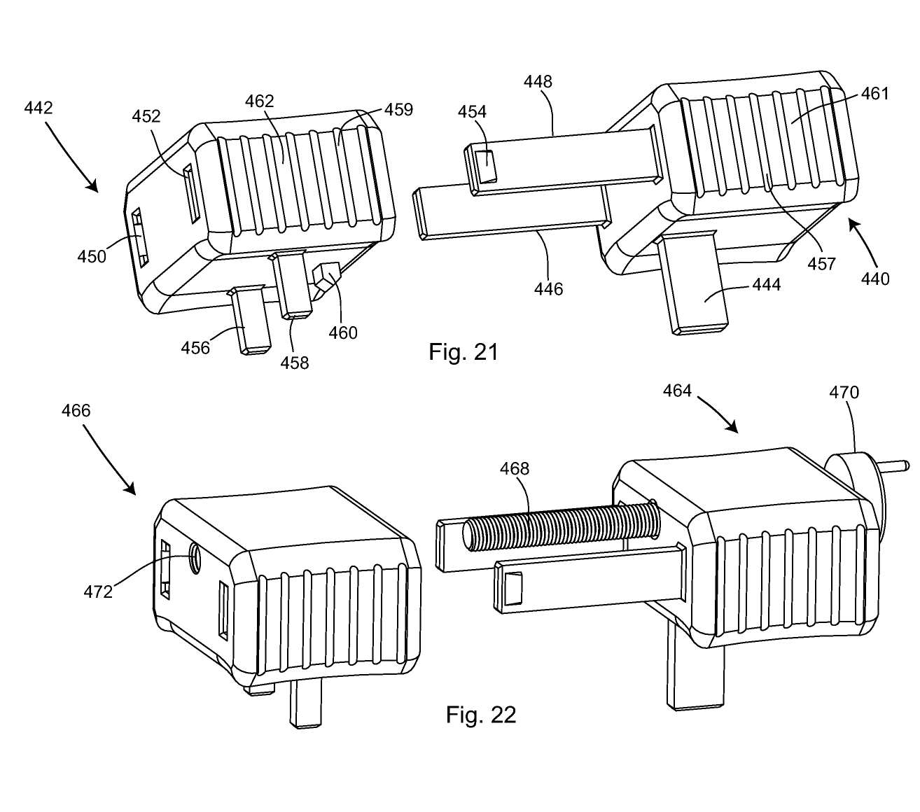

FIG. 22 shows an alternative embodiment of proximal and distal plugs (464), (466) comprising the same essential features as that of plugs (440), (442), mutatis mutandis, with the following differences. Distal plug (464) comprises an elongated screw (468) and a motor (470) for rotating screw (468) and mating with threaded cylindrical socket (472). Alternatively, screw (468) may be rotated manually.

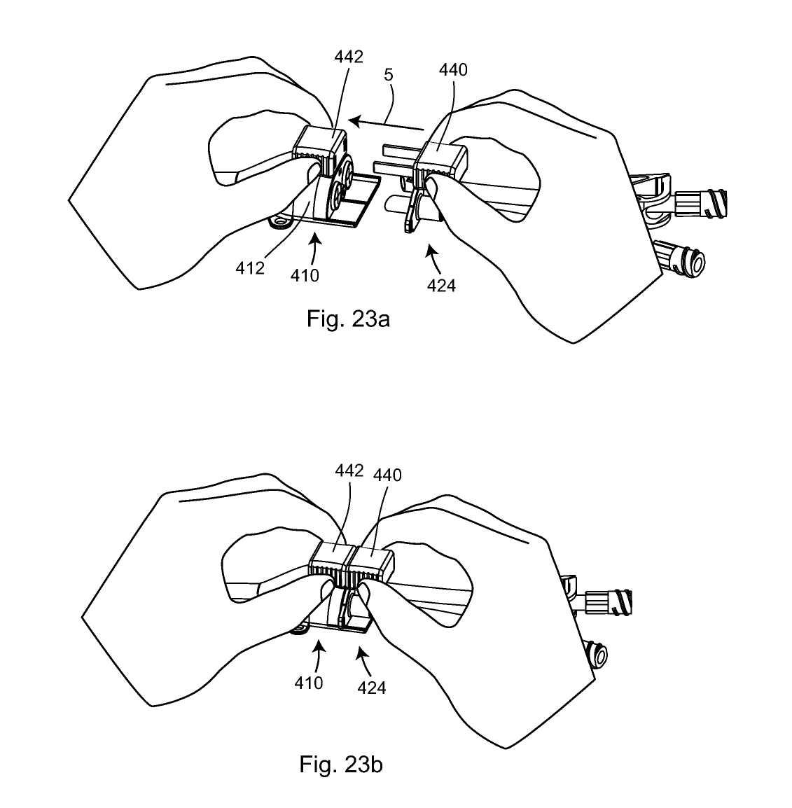

With reference to FIGS. 23a-b, showing the assembly process of connector (424) to hub (410), the transverse prong (444) (see FIG. 21) of plug (440) is inserted into the front cavity (436) (see FIG. 20c) of connector (424), and the transverse prongs (456), (458) of plug (442) are inserted into the front apertures (414), (416) of distal segment (412), as seen in FIG. 23a. Arrow (5) indicates the direction in which plug (440) is shifted in order to couple with plug (442). FIG. 23b shows plugs (440), (442) coupled together, and connector (424) attached to hub (410).

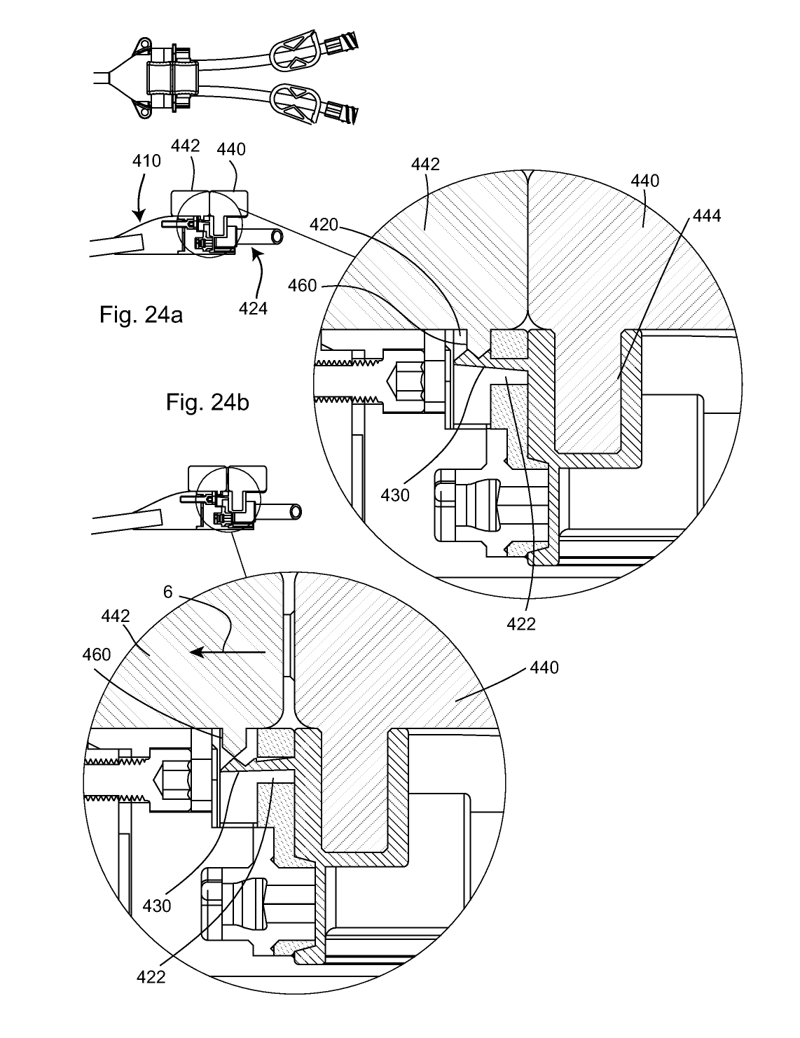

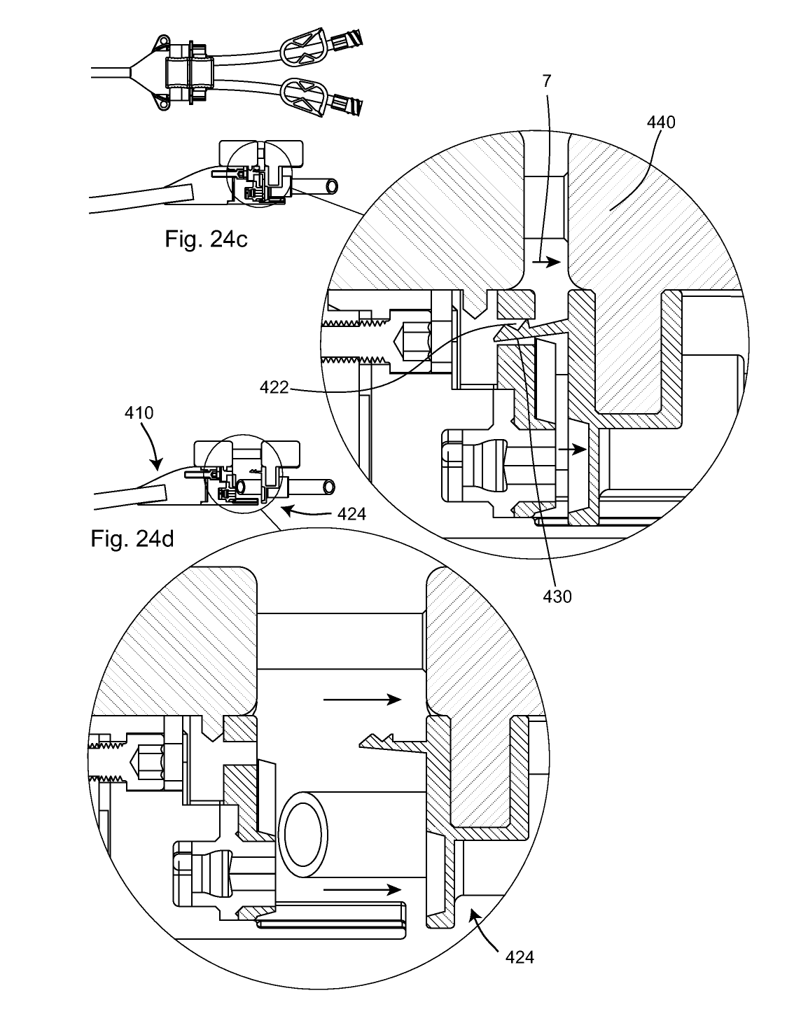

Referring to FIG. 24a, a cross-sectional side view of connector (424) secured to hub (410) is shown, and seen enlarged in detail showing transverse prong (444) of proximal plug (440) disposed within front cavity (436) of connector (424), tab (430) inserted through proximal aperture (422) and locking wedge (460) disposed within front aperture (420) and positioned between teeth of double toothed tab (430).

Referring to FIG. 24b, in order to unlock and remove connector (424) from hub (410) via plugs (440), (442), distal plug (442) is first shifted distally as indicated by arrow (6), thereby sliding locking wedge (460) out from between the teeth of tab (430) and shifting the teeth of tab (430) away from the wall (423) of proximal aperture (422).

FIG. 24c shows plug (440) shifted proximally, as indicated by arrow (7), thereby simultaneously shifting tab (460) out of proximal aperture (422). In FIG. 24d, connector (424) is separated from hub (410).

Plugs (464), (466) may be utilized to connect and disconnect connector (424) to and from hub (410), using the same method described herein above regarding plugs (440), (442), mutatis mutandis.

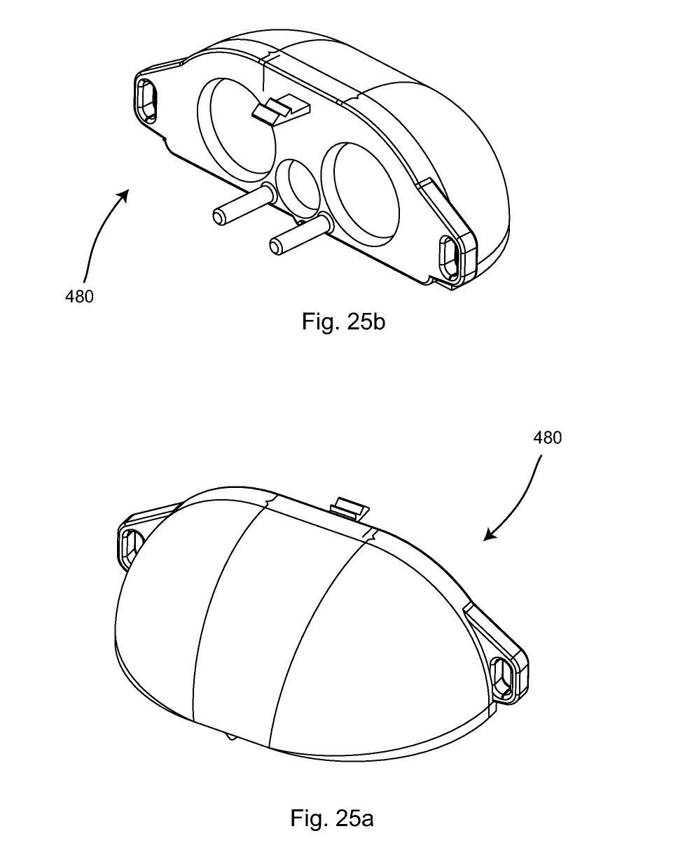

FIGS. 25a-b show an embodiment of an external connector (480), comprising a sealing cover for hub (410), having the same essential features as that of connector (424) in combination with sealing connector (240) for the first embodiment of device (10).

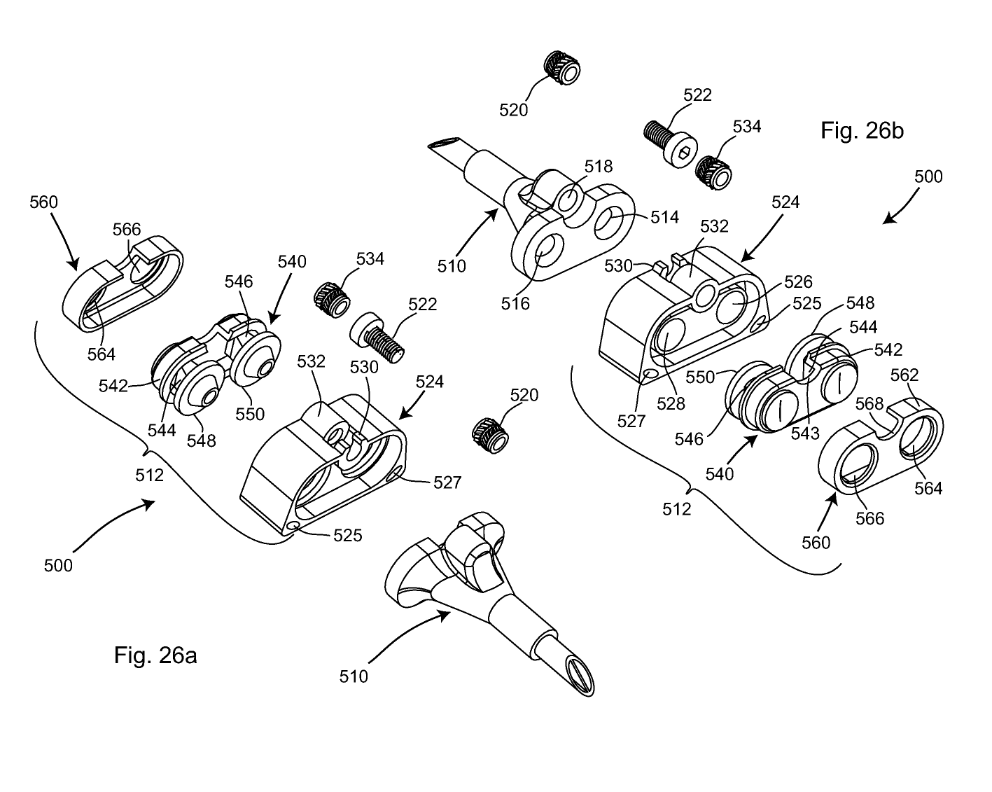

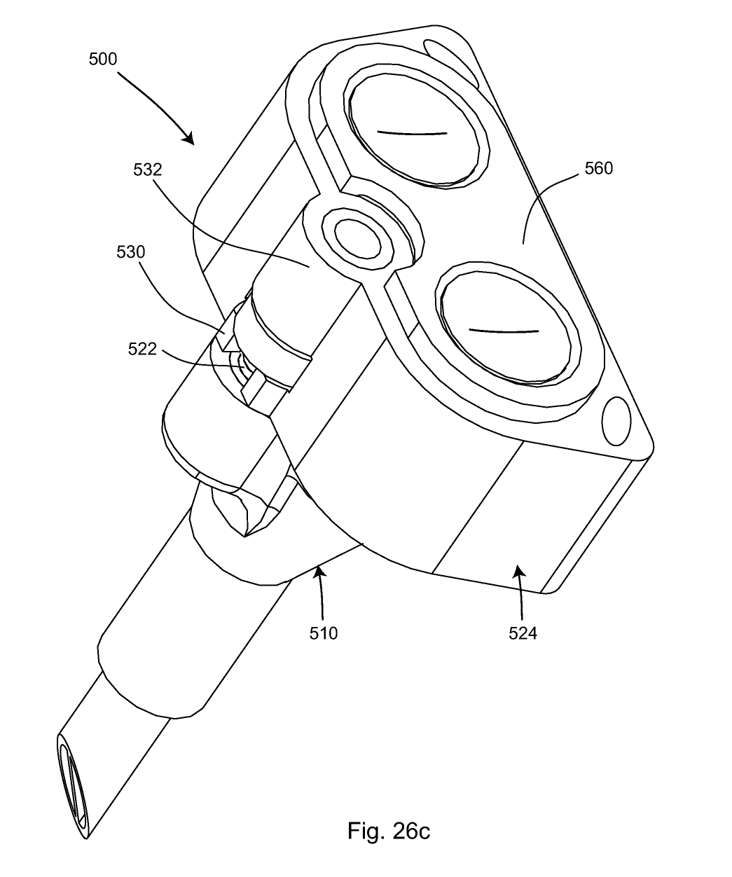

Referring to FIGS. 26a-b, a third embodiment of hub (500) is shown in a bottom exploded view (FIG. 26a) and a top exploded view (FIG. 26b). FIG. 26c shows an isometric assembled view of hub (500). Hub (500) comprises a molded catheter portion (510) and a valve housing portion (512) and comprises the same essential features and elements of the hub of the previous embodiments, mutatis mutandis, with the following differences.

Molded catheter portion (510) comprises lumens (514), (516) and a cavity (518) in which a distal threaded screw anchor (dowel) (520) is disposed for receiving bolt (522), as described herein below.

Valve housing portion (512) comprises a distal segment (524) having two lumens (526), (528), respectively aligned with lumens (514), (516) of molded catheter portion (510). The front side of distal segment (524) comprises a distal bolt holder (530) in the shape of an open ring for snapping bolt (522) therein, and a proximal channel (532) in which a proximal threaded screw anchor (dowel) (534) is disposed for receiving a bolt (not shown) for connecting an external connector to hub (500), as described herein below. Distal bolt holder (530) and proximal channel (532) are separated by a gap in which the head of bolt (522) is disposed.

Resilient flexible member (540) comprises a main body frame (542) for surrounding lumens (526), (528) of distal segment (524), having a u-shaped section (543) cut out of the front side for surrounding a portion of the wall of proximal channel (532) of distal segment (524). Valves (544), (546) extend distally from main body frame (542) and through lumens (526), (528). A circular gasket layer (548), (550) is situated around the distal portion of each valve (544), (546) for providing a tightly sealed relationship between distal segment (524) and molded catheter housing (510). The distal tip of each valve (544), (546) is disposed within one of each lumen (514), (516). Each valve (544), (546) also extends proximally from main body (542), having a proximal end comprising a slitted surface that is normally closed for disinfection swabbing.

Proximal segment (560) comprises a frame (562) for surrounding main body frame (542) of resilient flexible member (540) and two openings (564), (566) through which proximal portions of valves (544), (546) extend. A u-shaped section (568) is cut out of the front side of frame (562) for surrounding a portion of the wall of proximal channel (532) of distal segment (524).

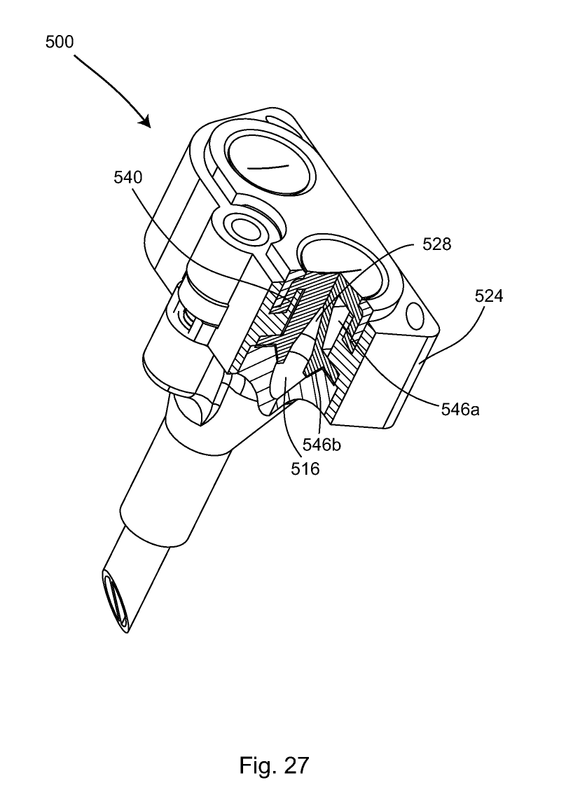

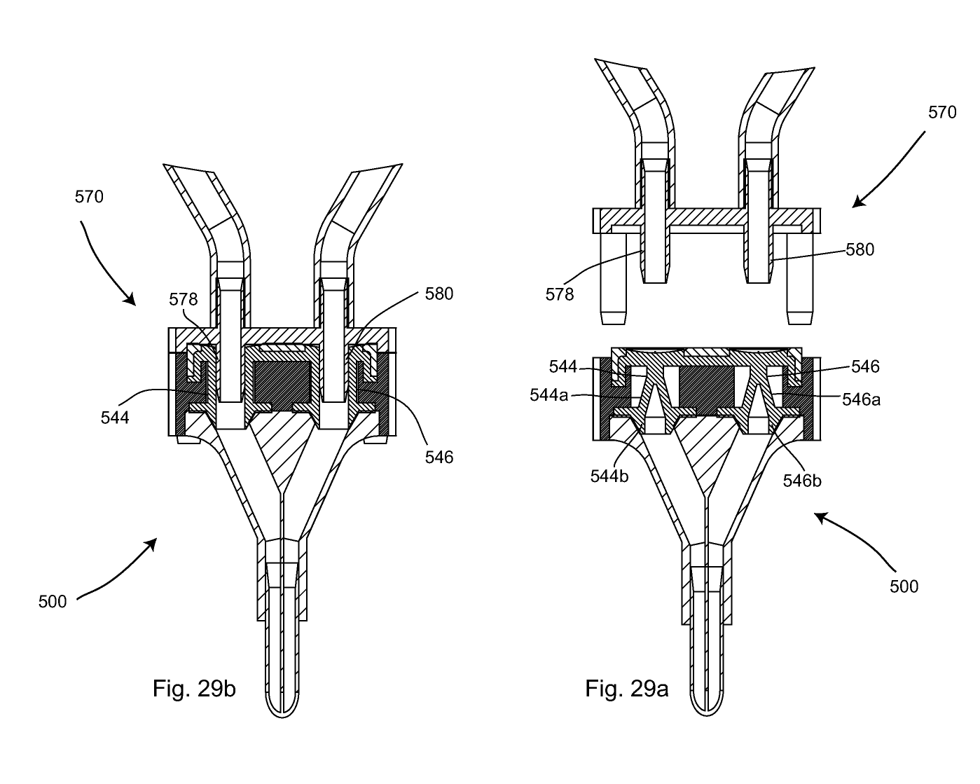

Referring to FIG. 27, hub (500) is shown assembled with a partial cross-sectional cut taken through lumens (528), (516), showing the intraluminal arrangement of resilient flexible member (540) and distal segment (524). In particular, and as seen best in FIG. 29a, distally extending valves (544), (546) comprise a proximal portion (544a), (546a) having a truncated wedge-shape, and a distal portion (544b), (546b) having a frusto-conical shape.

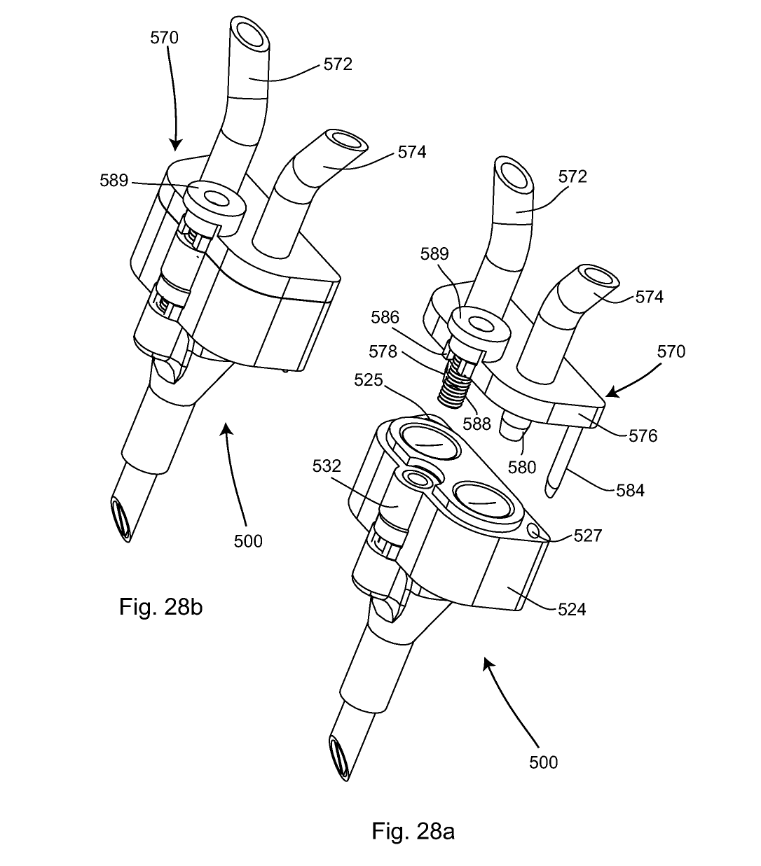

FIG. 28a shows a first embodiment of an external connector (570) comprising extension fluid lines (572), (574) in a top isometric view spaced apart from hub (500). Fluid lines (572), (574) extend proximally from main body (576), and cannulas (578), (580) extend distally from main body (576). Guiding pins (584) (only one shown) are inserted into openings (525), (527) (see FIGS. 26a-b) in distal segment (524). The front side of main body (576) comprises a proximal bolt holder (586) in the shape of an open ring for snapping bolt (588) therein. A standard screwdriver (not shown) may be used to screw bolt (588) into proximal channel (532), as shown in FIG. 28b. Bolt lock (589) integrally extends from the proximal side of main body (576) and extends over the head of bolt (588).

FIGS. 29a-b show a front cross-sectional view of hub (500) taken transversely through the lumens of hub (500) and connector (570), prior to joining connector (570) to hub (500) (FIG. 29a) and when connected to hub (500) (FIG. 29b). FIG. 29a shows the curvature of valves (544), (546) as described herein above. Upon insertion of cannulas (578), (580) into valves (544), (546) the hollow flow channel of valves (544), (546) takes essentially the shape of the tubular cannulas (578), (580). Cannulas (578), (580) are positioned partially, through valves (544), (546) such that a portion of valves (544), (546) extends past the distal tips of cannulas (578), (580).

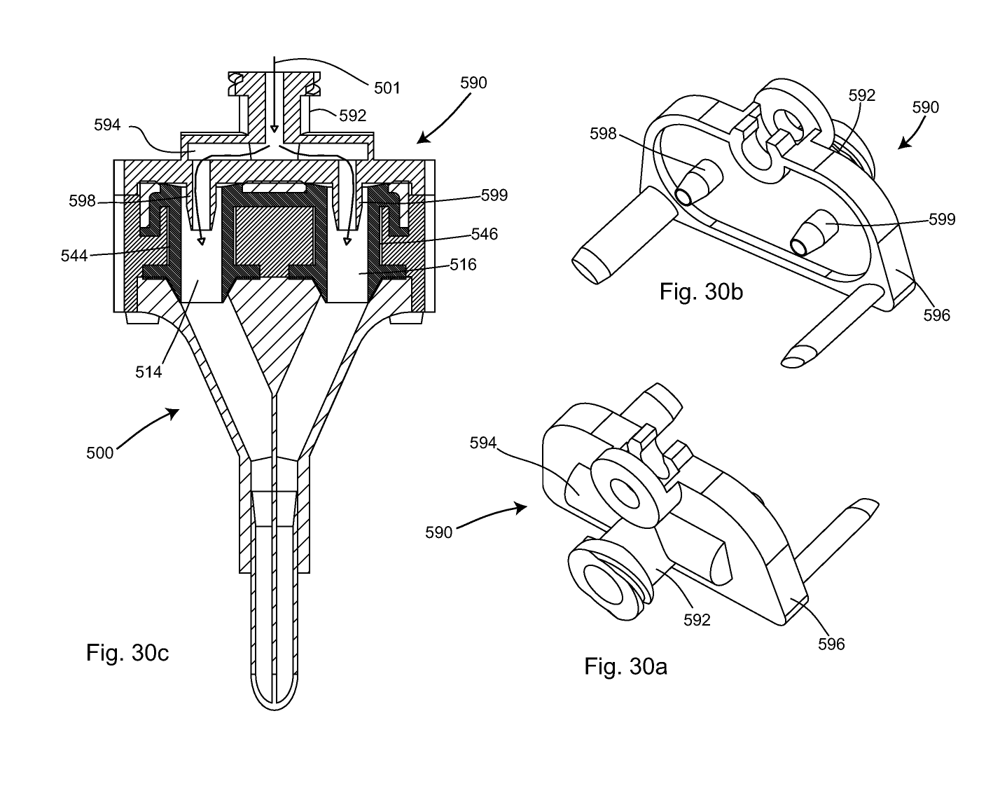

Another embodiment of an external connector (590) consisting of a flushing connector for hub (500) is shown in FIGS. 30a-c in a top isometric view (FIG. 30a), a bottom isometric view (FIG. 30b) and in a cross-sectional front view assembled with hub (500) (FIG. 30c). Connector (590) comprises the same essential features as that of connector (570) with the following differences. Female luer lock (592) extends longitudinally from the proximal side of connector (590) for flushing priming fluid through hub (500). Arrows (501) in FIG. 30c, indicate the path of fluid as it travels through female luer lock (592) and along conduit (594), which extends transversely along main body (596) of connector (590). The fluid travels from conduit (594) through cannulas (598), (599) and into valves (544), (546) disposed within lumens (514), (516).

Additional connectors such as a sealing connector (not shown) for sealing the valves of hub (500) and comprising the same essential features as that of the other connectors described for utilization with hub (500) may be used in the present invention, mutatis mutandis.

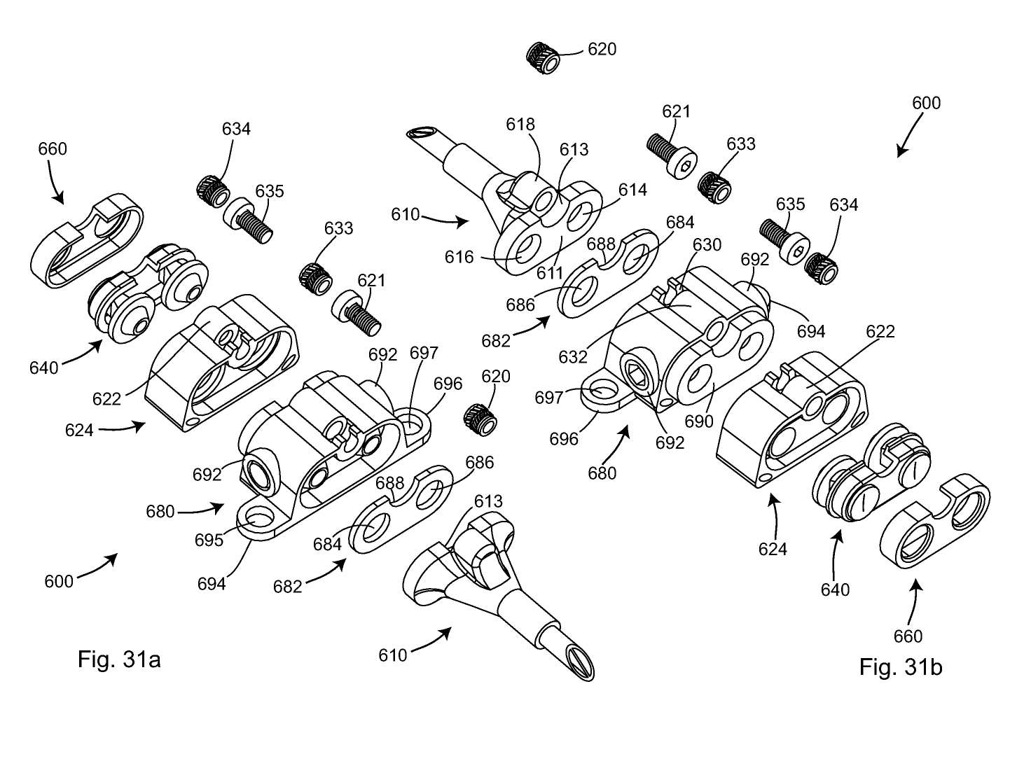

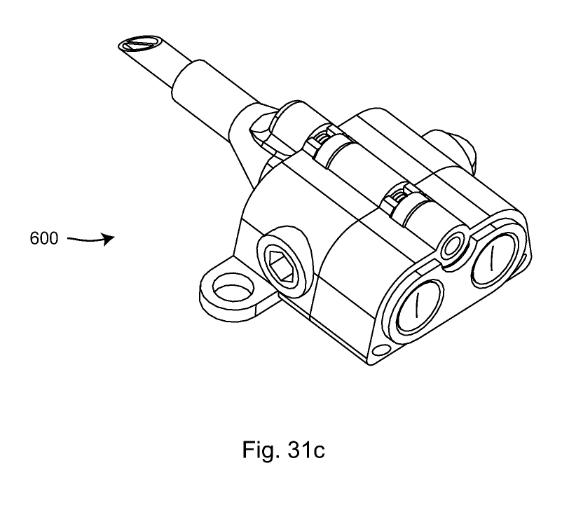

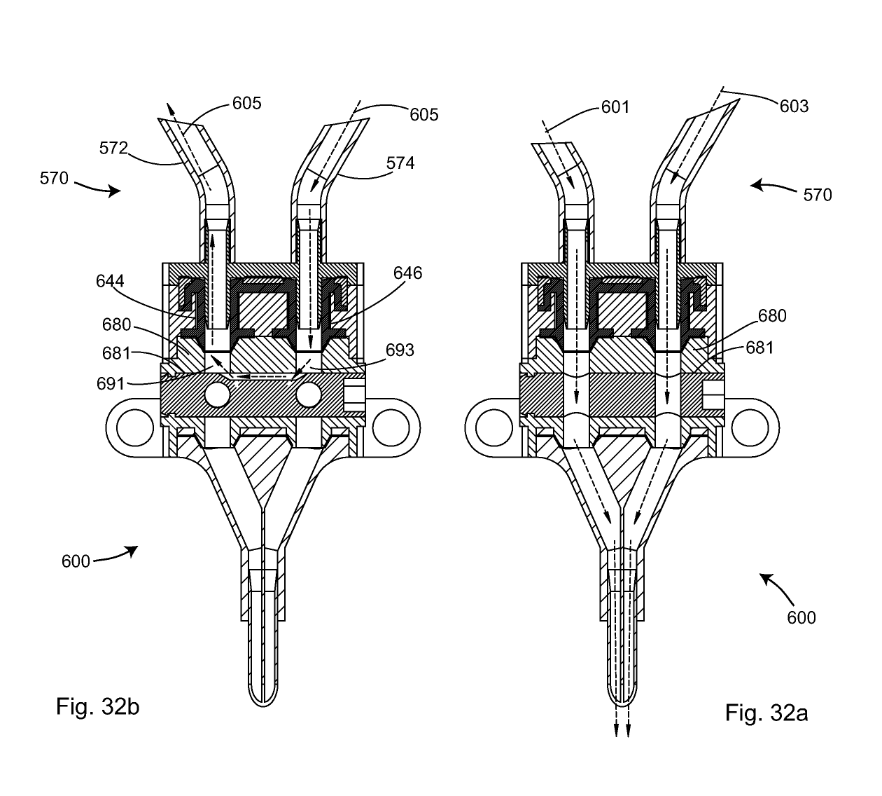

Referring to FIGS. 31a-b, a fourth embodiment of hub (600) is shown in a bottom exploded view (FIG. 31a) and a top exploded view (FIG. 31b). FIG. 31c shows an isometric assembled view of hub (600). Hub (600) comprises the same essential features and elements of the hub (500), mutatis mutandis, with the following differences. Hub (600) comprises a stopcock housing (680) having a stopcock mechanism (681) (see FIGS. 32a-b), and a gasket layer (682) situated between molded catheter portion (610) and distal segment (624).

Gasket layer (682) comprises the same shape as that of the proximal side (611) of molded catheter portion (610) such that the edges of gasket layer (682) align with the edges of proximal side (611) of molded catheter portion (610). Similarly, openings (684), (686) of gasket layer (682) are aligned with lumens (614), (616) of molded catheter portion (610), and the u-shaped section (688) cut out from the front edge of gasket layer (682) is aligned with the u-shaped section (613) cut out of the proximal front edge of molded catheter portion (610).

Stopcock housing (680) comprises a proximal side (690) over which distal segment (624) is positioned, and a front side comprising a distal bolt holder (630) in the shape of an open ring for snapping bolt (621) therein, and a proximal channel (632) in which a proximal threaded screw anchor (dowel) (633) is disposed for receiving a bolt (635) for connecting distal segment (624) thereto. Dowels (620), (634) are disposed within respective channels (618), (622). An actuating structure (692) is situated on at least one transverse side of stopcock housing (680), although in FIGS. 31a-b actuating structure (692) are situated at both transverse sides. In one preferred embodiment, actuating structure (692) is a female socket, such as a hex socket as shown in the figures. A standard hex screwdriver (or, hex wrench) may be used to rotate the internal stopcock mechanism between the open and closed positions, as seen in FIGS. 32a-b. Alternatively, a lever or knob (not shown) may be integral with actuating structure (692). Suturing ears (694), (696), having holes (695), (697) for attaching hub (600) to a patient (not shown), extend transversely from the rear wall of stopcock housing (680).

Referring to FIGS. 32a-b, external connector (570) is assembled with hub (600) and shown in a front cross-sectional view taken transversely through the lumens of hub (600) and connector (570). Stopcock mechanism (681) is shown in FIG. 32a in an open position for allowing fluid, indicated by arrows (601), (603) to flow unobstructed through hub (600). FIG. 32b shows stopcock mechanism (681) in a closed position for preventing fluid from flowing in one end of hub (600) and out the other end of hub (600) to be used for flushing valves (644), (646). Fluid, indicated by arrow (605) is shown entering one extension fluid line (574), travelling through valve (646) into the corresponding lumen (693) of stopcock housing (680), along the transverse duct (698) in stopcock mechanism (681), into the second lumen (691) of stopcock housing (680), through valve (644) to extension fluid line (572).

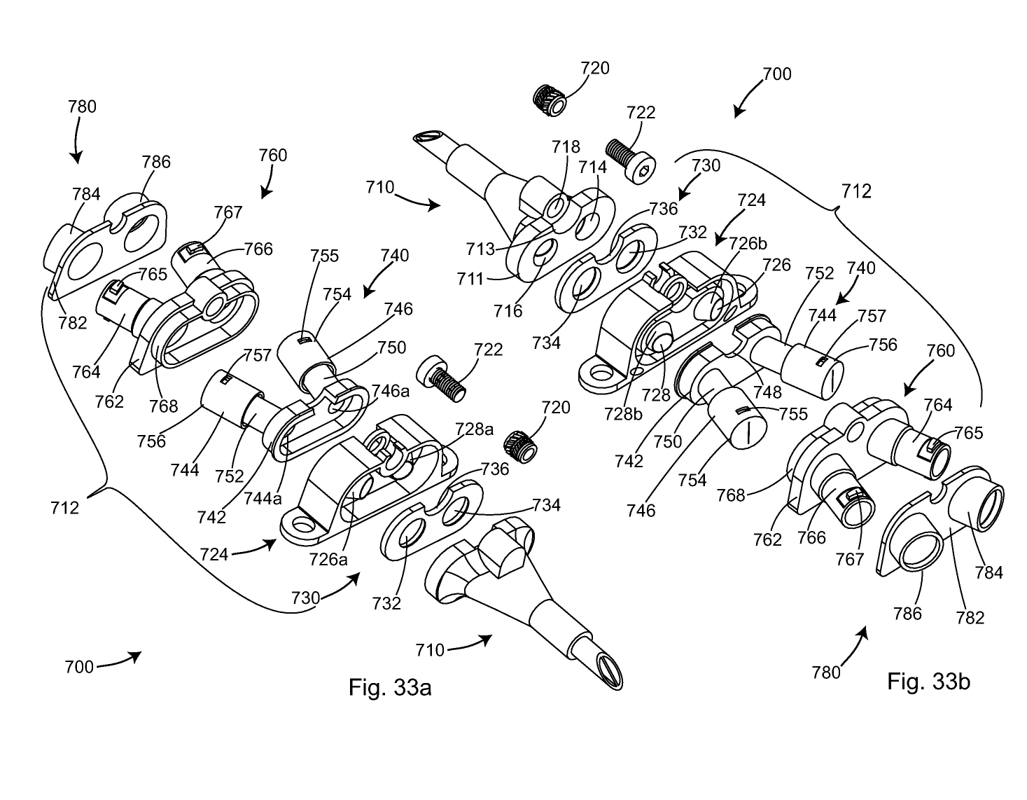

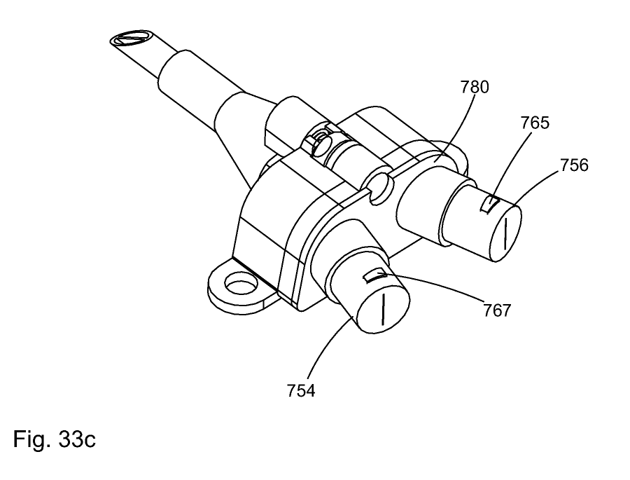

Referring to FIGS. 33a-b, a fifth embodiment of hub (700) is shown in a bottom exploded view (FIG. 33a) and a top exploded view (FIG. 33b). FIG. 33c shows an isometric assembled view of hub (700). Hub (700) comprises a molded catheter portion (710) and a valve housing portion (712) and comprises the same essential features and elements of hub 500, mutatis mutandis, with the following differences.

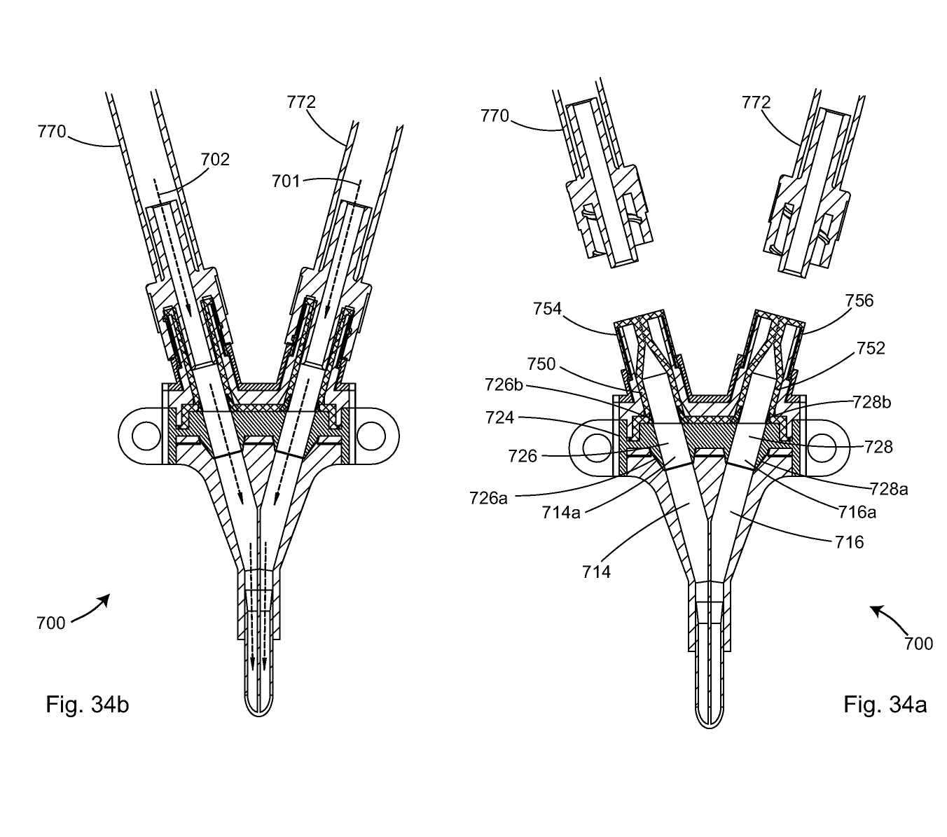

Molded catheter portion (710) comprises lumens (714), (716) and a cavity (718) in which a distal threaded screw anchor (dowel) (720) is disposed for receiving bolt (722). As best seen in FIG. 34a, showing a cross-sectional front view of assembled hub (700), openings (714a), (716a) of lumens (714), (716) are angled inward toward the central axis of hub (700) for receiving the angled lumens (726), (728) of distal segment (724) of valve housing portion (712), as described herein below.

Valve housing portion (712) comprises gasket layer (730) disposed between a distal segment (724) and molded catheter portion (710) for providing a sealed relationship and for preventing fluid leakage between distal segment (724) and molded catheter portion (710).

Gasket layer (730) comprises the same shape as that of the proximal side (711) of molded catheter portion (710) such that the edges of gasket layer (730) align with the edges of proximal side (711) of molded catheter portion (710). Similarly, openings (732), (734) of gasket layer (730) are aligned with lumens (714), (716) of molded catheter portion (710), and the u-shaped section (736) cut out from the front edge of gasket layer (730) is aligned with the u-shaped section (713) cut out of the proximal front edge of molded catheter portion (710).

Referring still to FIGS. 33a-b and FIG. 34a, distal segment (724) comprises lumens (726), (728) having a distally extending portion (726a), (728a) and a proximally extending portion (726b), (728b). Distally extending portion (726a), (728a) is angled toward the central axis of hub (700) for partial insertion into lumens (714), (716). Proximally extending portions (726b), (728b) are inserted partially into the distal openings (744a), (746a) of valves (744), (746) of resilient flexible member (740).

Resilient flexible member (740) comprises a main body (742) for surrounding lumens (726), (728) of distal segment (724), having a u-shaped section (748) cut out from the front side of main body (742). Valves (744), (746) angled inward toward the central axis of hub (700) extend proximally from main body (742), and comprise an elongated inner valve portion (750), (752) and an outer shell portion (754), (756) for covering a portion of proximal segment (760), described herein below.

Proximal segment (760) comprises a main body (762) and elongated tubular elements (764), (766) extending proximally therefrom in which inner valve portions (750), (752) of valves (744), (746) are disposed. Outer shell portions (754), (756) of valves (744), (746) cover the proximal portions of tubular elements (764), (766). Outer shell portions (754), (756) comprise apertures (755), (757) through which stopper elements (765), (767) situated near the proximal tip of tubular elements (764), (766) protrude, as best seen in FIG. 33c.

A transverse frame portion (768) extends distally from main body (762) of distal segment (760) for positioning over main body (742) of resilient flexible member (740). The front side of main body (762) comprises a channel through which a bolt may be threaded for attaching an external connector to hub (700). Proximal cover (780) comprises a main body (782) for positioning over main body (762) of proximal segment (760), and rings (784), (786) extending proximally from main body (762) through which valves (744), (746) and tubular elements (764), (766) protrude. Rings (784), (786) surround the distal edge of outer shell portions (754), (756) for restricting outer shell portions (754), (756) from bending outward.

FIGS. 34a-b show a cross-sectional front view of hub (700) with extension lines (770), (772) prior to connecting with hub (700) (FIG. 34a) and when connected with hub (700) (FIG. 34b). Extension lines (770), (772) are shown as male luer connectors, although any suitable form of connectors may be used. Arrows (701), (702) indicate fluid passing through the independent lumens of extension line (770), (772) and of hub (700) in FIG. 34b.

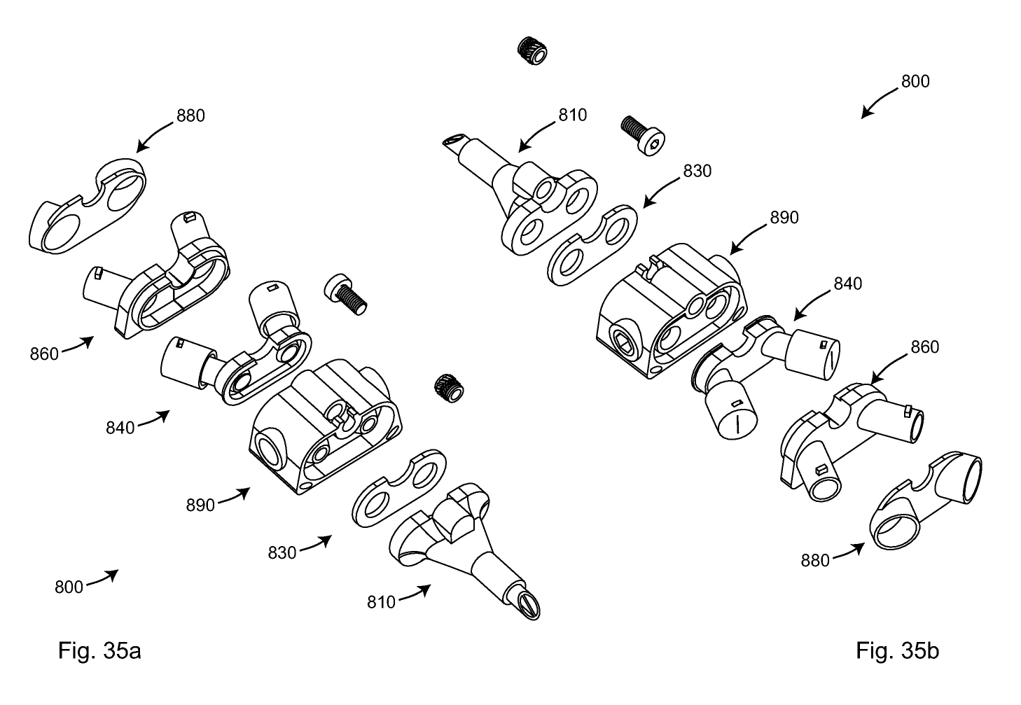

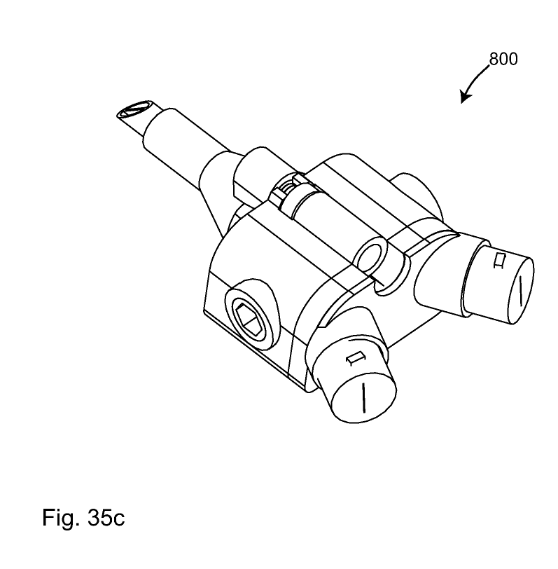

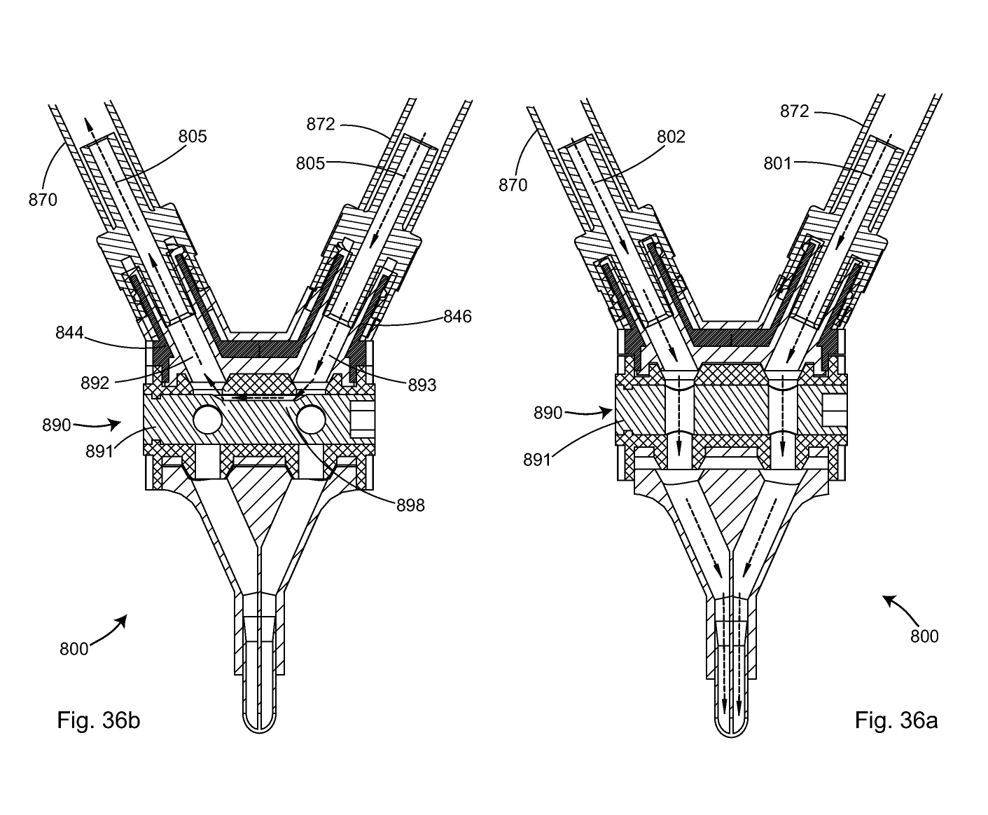

Referring to FIGS. 35a-b, a sixth embodiment of hub (800) is shown in a bottom exploded view (FIG. 35a) and a top exploded view (FIG. 35b). FIG. 35c shows an isometric assembled view of hub (800). Hub (800) comprises the same essential features and elements of the hub (700), mutatis mutandis, with the following differences. Hub (800) comprises a stopcock housing (890) having a stopcock mechanism (891) as best seen in FIGS. 36a-b (essentially the same as stopcock housing (680) and stopcock mechanism (681) of hub (600), mutatis mutandis), replacing distal segment (724) of hub (700).

Stopcock housing (890) comprises, with reference to FIGS. 31a and 31b, a proximal side (690) over which distal segment (624) is positioned, and a front side comprising a distal bolt holder (630) in the shape of an open ring for snapping bolt (621) therein, and a proximal channel (632) in which a proximal threaded screw anchor (dowel) (633) is disposed for receiving a bolt (635) for connecting distal segment (624) thereto. Dowels (620), (634) are disposed within respective channels (618), (622). Actuating structure (692) is situated on at least one transverse side of stopcock housing (680), although in FIGS. 31a-b actuating structure (692) are situated at both transverse sides. In one preferred embodiment, actuating structure is a female socket, such as a hex socket as shown in the figures. A standard hex screwdriver (or, hex wrench) may be used to rotate the internal stopcock mechanism between the open and closed positions, as seen in FIGS. 32a-b. Alternatively, a lever or knob (not shown) may be integral with actuating structure (692). Suturing ears (694), (696), having holes (695), (697) for attaching hub (600) to a patient (not shown), extend transversely from the rear wall of stopcock housing (680).

Referring to FIGS. 36a-b, external male luer connectors (870), (872) comprising extension fluid lines are assembled with hub (800) and shown in a front cross-sectional view taken transversely through the lumens of hub (800) and connectors (870), (872). Stopcock mechanism (891) is shown in FIG. 36a in an open position for allowing fluid, indicated by arrows (801), (802) to flow unobstructed through hub (800). FIG. 36b shows stopcock mechanism (891) in a closed position for preventing fluid from flowing in one end of hub (800) and out the other end of hub (800) to be used for flushing valves (844), (846). Fluid, indicated by arrow (805) is shown entering one extension fluid line (872), travelling through valve (846) into the corresponding lumen (893) of stopcock housing (890), along the transverse duct (898) in stopcock mechanism (891), into the second lumen (892) of stopcock housing (890), through valve (844) to extension fluid line (870).

While the invention has been described with respect to a limited number of embodiments, it will be appreciated that many variations, modifications and other applications of the invention may be made and will be evident to those of ordinary skill in the art without departing from the spirit and scope of the present invention as claimed.

1-43. (canceled)

44. An assembly comprising:
a. a port-like catheter hub designed to detachably engage with an external connector, said port-like catheter hub having at least two lumens extending therethrough and comprising of at least a molded catheter portion and a valve housing portion;
b. said molded catheter portion composed of
(i) a tapered molded body having a narrow tapered distal end and an enlarged proximal end, the narrow tapered distal end configured to one of receive and integrate with catheter tubing defining at least two lumens,
(ii) the enlarged proximal end terminating in a flat surface, with at least two openings defined in said flat surface, and
(iii) at least two bores extending from the openings through the molded body, with each bore at the distal end communicating with a distinctive lumen in a catheter;
c. said valve housing portion composed of
(i) a distal segment with at least two spaced through first openings,
(ii) a proximal segment with at least two spaced through second openings, and
(iii) a resilient gasket portion with at least two spaced first valves mounted between said proximal and distal segments,
(iv) with said at least first and second openings and at least first valves all in alignment and in alignment with the at least two bores extending from the flat surface of the proximal end of said molded catheter hub portion to define at least two separate fluid channels through said port-like catheter hub;
d. first structural elements operatively detachably cooperating to hold the molded catheter hub portion and valve housing portion together;
e. an external connector composed of one of an external connector mounting fluid lines for fluid communication, a sealing cover and a flushing or priming connector; and
f. second structural elements cooperating to hold the external connector in a fixed position relative to said port-like catheter hub.

45. The assembly according to claim 44 wherein a resilient sealing gasket is interposed between the distal segment of the valve housing portion and the molded catheter hub portion.

46. The assembly according to claim 44 further including a stopcock interposed and held between the catheter hub portion and the valve housing portion for controlling the separate fluid channels through said port-like catheter hub.

47. The assembly according to claim 44 wherein the port-like catheter hub further includes a back plate fixed to said distal segment that projects above the proximal segment and defines a guide groove in its surface adjacent to the proximal segment to engage with and guide the external connector to its fixed position relative to said port-like catheter hub.

48. The assembly according to claim 44 wherein a pair of spaced pins depend from the proximal segment that are received in holes defined in the distal segment and molded catheter hub portion to further guide and secure the port-like catheter hub.

49. The assembly according to claim 44 wherein said valve housing portion further includes a third opening in said proximal segment, positioned spaced from said first and second openings, providing fluid path access to said at least two separate fluid channels through said port-like catheter hub to serve as a flushing or priming valve;

50. The assembly according to claim 44 wherein the first and second structural elements are composed of one of a bolt arrangement and a plug arrangement.

51. The assembly according to claim 44 wherein the first and second structural elements include a first bolt to hold the molded catheter portion and valve housing portion together, and a second bolt to hold the external connector to the port-like catheter hub.

52. The assembly according to claim 51 wherein the second bolt is mounted on the first bolt via a flexible element that enables the second bolt to be loosened and bent outwardly to enable detachment of the external connector from the port-like catheter hub.

53. The assembly according to claim 44 wherein the external connector includes articulated arms terminating in hooks, biased toward one another, and the molded catheter portion of the assembly defines cutouts on opposite sides thereof to coact with the hooks and detachably hold the external connector securely in its fixed position relative to said port-like catheter hub.

54. The assembly according to claim 46 wherein the structural elements are comprised of a first set of a bolt and threaded element to hold the molded catheter portion and stopcock intercoupled together, a second set of a bolt and threaded element to hold the stopcock and valve housing portion intercoupled together, and a third set of a bolt and threaded element to hold the external connector and the valve housing portion intercoupled together.

55. The assembly according to claim 44 wherein the structural elements are comprised of a first plug with prongs for insertion into at least one slot in said molded catheter portion, and a second plug with prongs for insertion into at least one slot in said external connector, and said second plug having at least one prong for insertion into at least one slot in said first plug.

56. The assembly according to claim 55 wherein the first plug has a through bore providing a threaded socket facing the second plug and the second plug has a threaded shaft that is one of manually or motor driven for being received in said through-bore in said first plug and intermeshing with the threaded socket.

57. The assembly according to claim 44 wherein each of the at least two spaced first valves of said port-like catheter hub comprise a proximal portion having a truncated wedge-shape, and a distal portion having a frusta-conical shape.

58. The assembly according to claim 44 wherein the at least two spaced valves of the valve housing portion are configured as female Iuers for connection to a pair of conduits that terminate with male Iuers mounted on the external connector and wherein said at least two separately defined fluid channels through said port-like catheter hub are angularly disposed in a non-parallel arrangement.

59. A method for providing at least two lumens of catheter tubing relative to a human body to administrate therapeutic fluids, drugs or blood in an inlet/outlet dialysis procedure where blood is extracted via one lumen and is returned to the body via another lumen of the catheter tubing comprising the steps of:
a. operatively inserting, via the skin of a human into a blood vessel of the human, a multi-lumen catheter tubing having at least two lumens;
b. providing a molded catheter portion composed of a tapered molded body having a narrow tapered end and an enlarged other end, the narrow tapered end configured to couple with catheter tubing defining at least two lumens, the enlarged other end terminating in a flat surface, with at least two openings defined in said flat surface and at least two bores extending from the openings through the molded body,
c. connecting each bore of the molded catheter portion at the distal end thereof with a distinct lumen of the multi-lumen catheter tubing;
d. providing a valve housing portion composed of a distal segment with at least two spaced through first openings, a proximal segment with at least two spaced through second openings and a resilient gasket portion with at least two spaced first valves mounted between said proximal and distal segments;
e. intercoupling detachably the molded catheter portion and valve housing portion together by first structural elements operatively and cooperatively holding the portions together with said at least first and second openings and first valves all in alignment and in alignment with the at least two bores extending from the flat surface of the molded catheter portion to define at least two separate fluid channels through said intercoupled molded catheter portion and valve housing portion;
f. fixing the molded catheter portion to the skin of the body adjacent to tubing insertion;
g. intercoupling detachably an external connector to the intercoupled molded catheter portion and valve housing portion by second structural elements operatively and cooperatively holding the external connector in a relatively fixed position to the intercoupled molded catheter portion and valve housing portion, said external connector mounting at least two fluid extension lines;
h. connecting distal ends of the at least two fluid extension lines to the at least two separate fluid channels via cannulas protruding into the valve housing portion of said intercoupled molded catheter portion and valve housing portion via the at least two spaced first valves of the resilient gasket portion; and
i. coupling proximal ends of the at least two fluid extension lines to a device for administration of therapeutic fluids, administration of drugs or processing blood in an inlet/outlet dialysis procedure.

60. The method according to claim 59 including the further steps of
j. providing the molded catheter portion with a back plate having a guiding element; and
k. effecting the intercoupling of the external connector and the intercoupled molded catheter portion and valve housing portion by guiding them together via mutually coacting guiding elements.

61. The method according to claim 59 including the further steps of
l. providing the valve housing portion with a third opening in said proximal segment positioned between said at least first and second openings and a second valve on said resilient gasket portion in alignment with said third opening to serve as a priming or flushing valve; and
m. introducing priming or flushing fluid into said third opening to prime or flush the molded catheter portion and the valve housing portion.

62. The method according to claim 59 including the further steps of
n. detaching the external connector from the molded catheter portion and valve housing portion;
o. detaching the intercoupled the molded catheter portion and valve housing portion;
p. replacing the valve housing portion with a new valve housing portion, and
q. intercoupling detachably the new valve housing portion with the molded catheter portion; and
r. intercoupling detachably the external connector and the molded catheter portion and new valve housing portion.

63. The method according to claim 59 including the further steps of
s. interrupting the method of claim 59;
t. detaching the external connector from the molded catheter portion and valve housing portion so the valve housing portion is exposed;
u. intercoupling detachably an external connector in the form of a sealing cover to the molded catheter portion and exposed valve housing portion to cover and protect the valve housing portion from contamination and undesirable mechanical interference; and
v. after a predetermined interval, detaching the external connector in the form of a sealing cover and intercoupling detachably an external connector mounting at least two fluid extension lines and continuing the method of claim 59.
